(19) United States
(12) Patent Application Publication
(10) Pub. No.: US 2022/0175870 A1
(43) Pub. Date: Jun. 9, 2022

(54) Therapeutic Compositions Directed To Host Mirna For The Treatment Of Sars-Cov-2 (Covid-19) Infection

(71) Applicants: The Regents of the University of Colorado, a body corporate, Denver, CO (US); United States of America as Represented by the Administrator of NASA, Washington, DC (US)

(72) Inventors: Anushree Chatterjee, Boulder, CO (US); Afshin Beheshti, Medford, MA (US); Sylvain Vincent Costes, Albany, CA (US); Thomas R. Aunins, Boulder, CO (US); Prashant Nagpal, Lafayette, CO (US)

(21) Appl. No.: 17/541,737

(22) Filed: Dec. 3, 2021

Related U.S. Application Data
(60) Provisional application No. 63/121,885, filed on Dec. 5, 2020.

(51) Int. Cl. A61K 38/04

(52) U.S. Cl. CPC A61K 38/04

(57) ABSTRACT

The inventive technology generally relates to systems, methods, and compositions for the treatment of viral infections, as well as novel use of antisense technology to rationally design antiviral compositions that can be applied to clinical cases and human infections. In one preferred aspect, the inventive technology includes methods, and compositions to treat COVID-19 in humans through the targeted inhibition of host-derived miRNAs.

### CROSS REFERENCES TO RELATED APPLICATION

This application claims the benefit of and priority to U.S. Provisional Application No. 63/121,885, filed Dec. 5, 2020. The entire specification and figures of the above-referenced application is hereby incorporated in its entirety by reference.

### STATEMENT OF FEDERALLY SPONSORED RESEARCH

This invention was made with government support under grant number NNX16A069A awarded by NASA. The government has certain rights in the invention.

### SEQUENCE LISTING

The instant application contains a Sequence Listing which has been submitted electronically in ASCII format and is hereby incorporated by reference in its entirety. Said ASCII copy, created on Dec. 3, 2021, is named “90245-00571-Sequence-Listing-AF” and is 1.12 Kbytes in size.

### TECHNICAL FIELD

The inventive technology includes novel systems, methods, and compositions for the treatment and prevention of infection by the novel SARS-CoV-2 coronavirus (COVID-19). In particular, the inventive technology includes methods of generating, and compositions for targeting host microRNAs (miRNAs) for the treatment of COVID-19.

### BACKGROUND

In 2019, a novel coronavirus identified as COVID-19, having a high infection and mortality rate, emerged in the Wuhan region of China, and later spread throughout the world resulting in sever public health crisis. Coronaviruses, members of the Coronaviridae family and the Coronavirinae subfamily, are found in mammals and birds. A prominent member is severe acute respiratory syndrome coronavirus (SARS-CoV), which killed almost 10% of the affected individuals during an outbreak in China between 2002 and 2003. Another prominent coronavirus called Middle East Respiratory Syndrome Coronavirus (MERS coronavirus or MERS-CoV) MERS-CoV shares some similarities with the SARS-CoV outbreak. Typical symptoms of a SARS. MERS and COVID-19 coronavirus infection include fever, cough, shortness of breath, pneumonia and gastrointestinal symptoms. Severe illness can lead to respiratory failure that requires mechanical ventilation and support in an intensive care unit. Both coronavirus appears to cause more severe disease in older people, people with weakened immune systems and those with chronic diseases, such as cancer, chronic lung disease and diabetes. At present no approved vaccine or specifically effective treatments are available for COVID-19. Patients diagnosed with a COVID-19 coronavirus infection merely receive supportive treatment based on the individual's symptoms and clinical condition.

Prior research on the interactions between microRNAs (miRNAs) and viruses have revealed a complex interaction that may show promise as a potential therapeutic target for the prevention of viral infections, and in particular COVID-19. Specifically, viruses have been shown to avoid the immune response by making use of cellular miRNAs to finish their replication cycles. The following mechanisms have been shown to be central in the interaction of viruses and miRNAs: 1) miRNA pathways are blocked or inhibited by viruses interacting with key proteins, 2) viruses may employ specific target mRNAs to avoid or dysregulate cellular miRNAs, 3) viruses can utilize miRNAs to redirect regulatory pathways to other miRNA targets to provide survival advantages, and viruses can encode miRNAs to produce viral miRNAs with well-defined functions to specifically target and regulate functions related to that virus. Individual miRNAs can target multiple messenger RNAs (mRNAs) and are predicted to regulate over half of the human transcriptome. Recent evidence has shown that there are distinct miRNAs in the blood that arises from different health risks, which includes SARS-CoV-2 (COVID-19) infection. These circulating miRNAs are highly stable, resistant to degradation, and have potential to be used as a minimally invasive novel detection and therapeutic strategy.

Due to the novel nature of COVID-19 and the lack of understanding of the key biological mechanisms of how this virus interacts with the host, methods to stop the viral infection from impacting the host are lacking. This complex nature of this virus provides unique issues with standard approaches to tackle viral research for development of a vaccine. One novel approached described herein involves the identification of novel miRNAs that drive COVID-19 replication and propagation in a human host, coupled with the rational design and synthesis of antisense inhibitors of these miRNAs and their associated therapeutic uses to treat COVID-19.

### SUMMARY OF THE INVENTION

The inventive technology generally relates to systems, methods, and compositions for the treatment of viral infections, as well as novel use of antisense technology to rationally design antiviral compositions that can be applied to clinical cases and human infections. In one preferred aspect, the inventive technology includes methods, and compositions to treat COVID-19 in humans through the targeted inhibition of host-derived miRNAs.

One aspect of the inventive technology includes the rational design and use of antisense therapeutics configured to target host-derive miRNA, and in particular host-derived miRNAs involved in viral infections. In one preferred aspect, the inventive technology includes the rational design and use of antisense FASTmers configured to target host-derived miRNA involved in SAR-CoV-2 infection, specifically, and in particular host-derived miRNA-2392 (also sometimes referred to as miR-2392, and identified herein as SEQ ID NO. 4). In another preferred aspect, the inventive technology includes the rational design and use of antisense FASTmers, such as antisense peptide-nucleic acid (PNA) FASTmer therapeutics configured to target host-derived miRNA involved in SAR-CoV-2 infection, and in particular host-derived miRNA-2392.

Another aspect of the inventive technology includes the use of rationally designed antisense PNA therapeutics to treat SAR-CoV-2 infection in a subject. In another aspect, the inventive technology includes the co-administration of rationally designed antisense PNA therapeutics with other therapeutic compositions to treat SAR-CoV-2 infection in a subject.

In another aspect of the inventive technology includes the prophylactic use of rationally designed antisense PNA therapeutics to prevent SAR-CoV-2 infection in a subject at risk of infection. In still further aspects, the inventive technology includes the prophylactic co-administration of rationally designed antisense PNA therapeutics with other prophylactic therapeutic compositions, such as vaccines, to prevent SAR-CoV-2 infection in a subject at risk of infection.

Another aspect of the invention includes antisense FASTmers configured to be complementary to a target sequence of miRNA-2392 in a host. Another aspect of the invention include antisense FASTmers configured to be complementary to a target sequence of miRNA-2392 in a host, and wherein said antisense FASTmers comprise antisense PNAs according to SEQ ID NOs. 1-2. Another aspect of the invention includes the isolated PNAs sequences according to SEQ ID NOs. 1, and 2.

Another aspect of the invention includes pharmaceutical compositions containing one or more antisense FASTmers configured to inhibit one or more host-derived miRNAs. In one preferred aspect, the invention includes methods of treating COVID-19 in a subject in need thereof, comprising the step of administering a therapeutically effective amount of a pharmaceutical compositions containing one or more antisense FASTmers configured to inhibit one or more host-derived miRNAs.

The inventive technology further relates to a composition comprising an antisense FASTmers, and preferably an antisense PNA FASTmer and the use of the composition for the preparation of a pharmaceutical composition, especially a therapeutic compound, e.g., for use in the prophylaxis or treatment of COVID-19 coronavirus infection. The inventive technology further describes methods of treatment or prevention of infections of COVID-19 coronavirus in subjects in need thereof using the composition comprising an antisense FASTmers, and preferably an antisense PNA FASTmer.

Another aspect of the invention includes pharmaceutical compositions containing one or more antisense PNA FASTmers configured to inhibit miRNA-2392. In one preferred aspect, the invention includes methods of treating COVID-19 in a subject in need thereof, comprising the step of administering a therapeutically effective amount of a pharmaceutical compositions containing one or more antisense PNA FASTmers configured to inhibit miRNA-2392.

Another aspect of the invention includes the identification of novel miRNA targets for the treatment of viral infection, and in particular novel host-derived miRNA targets for the treatment of COVID-19. Another aspect of the invention includes the rational design of novel miRNA inhibitors that may be used for the treatment of viral infection, and in particular novel inhibitors of host-derived miRNA for the treatment of COVID-19.

Additional aspects of the invention may be evidenced from the specification, claims and figures provided below.

### BRIEF DESCRIPTION OF DRAWINGS

The novel aspects, features, and advantages of the present disclosure will be better understood from the following detailed descriptions taken in conjunction with the accompanying figures, all of which are given by way of illustration only, and are not limiting the presently disclosed embodiments, in which:

FIG. 1: shows the Facile Accelerated Specific Therapeutic (FAST) platform strategy that generates antivirals compositions (FASTmers) against SARS-CoV2 with an accelerated design, build, test cycles of less than a week.

FIG. 2: shows an exemplary FASTmer targeting human miRNA-2392.

FIG. 3: shows the treatment of Vero cells infected with SARS-Cov2 at a MOI of 0.01.

FIG. 4: shows the treatment of Vero cells infected with SARS-Cov2 at a MOI of 0.01.

FIG. 5: shows the Evaluation of toxicity and efficacy of FASTmer in an in vivo infection model.

FIG. 6: shows the impact miR-2392 compared to other identified miRNAs in COVID-19 positive patients compared to negative patients and the related pathways that miR-2392 impacts.

FIG. 7: shows impact of the miR-2392 on the host during COVID-19 infection by utilizing RNA-sequencing data from nasal pharyngeal swabs of COVID-19 positive and negative patients. Figure specifically demonstrates the regulation of downstream miR-2392 gene targets indicating that miR-2392 targets are heavily involved as a function of viral load during COVID-19 infections.

FIG. 8: shows the highly conserved nature of miR-2392 across different species.

### DETAILED DESCRIPTION OF INVENTION

Generally, the inventive technology includes the use of predictive homology to rationally design novel antiviral compositions. In one preferred embodiment, the invention includes the identification of one or more miRNAs expressed in a host in response to a COVID-19 coronavirus infection that may be involved in propagating the viral infection. Target sequences are identified in the target miRNAs, which may preferably include sequences that are conserved across one or more species known to be susceptible to COVID-19 infection. Once a target sequence has been identified, the invention further includes systems and methods of rational designing one or more antagomirs complementary to the miRNA target sequence, which bind to, and inhibit the host miRNAs activity. These antagomirs are also referred to as inhibitors, or miRNA inhibitors, or “FASTmers” as described below, and may include one or more antisense-oligonucleotides.

In one preferred embodiment, the miRNA inhibitor or “FASTmer” of the invention may include an antisense peptide nucleic acid (PNA) molecule designed to inhibit host miRNA activity. as well as downstream expression of genes that may be influenced by said miRNA. Inhibition of host miRNA activity also inhibits downstream gene expression of one or more genes in a host or virus. As used herein, PNAs may be DNA analogs in which the phosphate backbone has been replaced by (2-aminoethyl) glycine carbonyl units that are linked to the nucleotide bases by the glycine amino nitrogen and methylene carbonyl linkers. The backbone is thus composed of peptide bonds linking the nucleobases. Because the PNA backbone is composed of peptide linkages, the PNA is typically referred to as having an amino-terminal and a carboxy-terminal end. However, a PNA can be also referred to as having a 5′ and a 3′ end in the conventional sense, with reference to the complementary nucleic acid sequence to which it specifically hybridizes. The sequence of a PNA molecule is described in conventional fashion as having nucleotides G, U, T, A, and C that correspond to the nucleotide sequence of the DNA molecule. Such polynucleotides can be synthesized, for example, using an automated DNA synthesizer. Typically, PNAs are synthesized using either Boc or Fmoc chemistry. PNAs and other polynucleotides can be chemically derivatized by methods known to those skilled in the art. For example, PNAs have amino and carboxy groups at the 5′ and 3′ ends, respectively, that can be further derivatized. Custom PNAs can also be synthesized and purchased commercially. Since PNA is structurally markedly different from DNA, PNA is very resistant to both proteases and nucleases, and is not recognized by the hepatic transporter(s) recognizing DNA. However, it should be noted that in one preferred embodiment a FASTmer inhibitor is a PNA oligomer directed to a target sequence of a miRNA, in alternative embodiments a FASTmer may include any antisense, or other oligomer that may bind to and inhibit miRNA activity in a host.

One embodiment of the inventive technology includes the rational design and use of antisense therapeutics configured to target host-derived miRNA, and in particular host-derived miRNAs involved in viral infections. In another preferred embodiment, the inventive technology includes the rational design and use of antisense FASTmers, such as antisense peptide-nucleic acid (PNA) FASTmer therapeutics configured to target host-derived miRNA involved in SAR-CoV-2 infection, and in particular host-derived miRNAs selected from the group consisting of: miRNA-181a-5p, miRNA-10, miRNA-10a-5p, miRNA-2393, miRNA-1-5p, miRNA-34a-5, miRNA-30c-5p, miRNA-29b-3p, miRNA-155-5p, and miRNA-124-3p. In one preferred embodiment, the inventive technology includes the rational design and use of antisense FASTmers configured to target host-derived miRNA involved in SAR-CoV-2 infection, specifically, and in particular host-derived miRNA-2392 (also sometimes referred to as miR-2392).

In some embodiments, a PNA FASTmer for treating a COVID-19 coronavirus infection includes at least one, at least two, at least three, or at least four or more antisense PNAs FASTmers configured to inhibit miRNA in a host, wherein each antisense PNA FASTmer includes a sequence of at least 5, and up to 20 or more nucleic acids capable of hybridizing to a target sequence of a miRNA. In some embodiments, the antisense PNA molecules of the PNA system may be configured to target a conserved region of a miRNA, or a combination thereof. It will be recognized by those of skill in the art that any of the DNA or RNA sequences described above can be targeted by antisense inhibitors. Target sequences can be those of a host-derived miRNA, DNA sequence that expresses a target miRNA, or a viral DNA or RNA sequence. Given the benefit of this disclosure, those of skill in the art will be able to identify a target sequence and design an antisense inhibitor oligomer to target the DNA or miRNA sequence. Inhibition can be caused by steric interference resulting from an antisense oligomer binding the target sequence, thereby preventing the targets function or activity, as well as expression of downstream genes that are responsive to, for example a target miRNA such as miRNA-2391. Target sites, such as those identified as SEQ ID NO. 3, can be any site to which binding of an antisense oligomer will inhibit transcription of the miRNA.

An antisense FASTmer oligomer can be complementary to a single target sequence or to two or more target sequence. Alternatively, as shown in Table 1, multiple antisense FASTmer oligomers, such as α-miR2392 (SEQ ID NO. 1 and SEQ ID NO. 2) can target a single target sequence. In particular embodiments, each individual antisense oligomer is complementary to a single target site. Wherein each individual antisense oligomer is complementary to a single target site, the antisense oligomer can be about less than 10-mers, 10-mers to 20-mers in length, or greater than 20-mers. In certain embodiments, the antisense FASTmer oligomer is 15-mers in length. In certain embodiments, the antisense inhibitory oligomers are designed with the target sequence in the middle of the oligomer, with 3-5 nucleotides flanking the overlapping region. This provides for antisense oligomers with both high affinity and specificity. Wherein an individual antisense oligomer is complementary to two or more target sites, the antisense oligomer can be up to about 40-mers or greater in length.

Another embodiment of the inventive technology includes the use of rationally designed antisense PNA therapeutics to treat SAR-CoV-2 infection in a subject. In another embodiment, the inventive technology includes the co-administration of rationally designed antisense PNA therapeutics with other therapeutic compositions to treat SAR-CoV-2 infection in a subject. In yet another embodiment, of the inventive technology includes the prophylactic use of rationally designed antisense PNA therapeutics to prevent SAR-CoV-2 infection in a subject at risk of infection. In still further embodiments, the inventive technology includes the prophylactic co-administration of rationally designed antisense PNA therapeutics with other prophylactic therapeutic compositions, such as vaccines, to prevent SAR-CoV-2 infection in a subject at risk of infection. Another embodiment of the invention includes antisense FASTmers configured to be complementary to a target sequence of miRNA-2392 in a host. Another embodiment of the invention include antisense FASTmers configured to be complementary to a target sequence of miRNA-2392 in a host, and wherein said antisense FASTmers comprise antisense PNAs according to SEQ ID NOs. 1-2.

A composition of the present disclosure can comprise one or more antisense FASTmers oligomers, such as a rationally designed antisense PNA configured to inhibit one or more miRNAs and may further have antiviral therapeutic effects. For example, a composition can comprise an antisense PNA FASTmers according to SEQ ID NOs 1-2, directed to SEQ ID NO. 4, and in particular the target sequence of SEQ ID NO. 4, identified as SEQ ID NO. 3. Another embodiment of the invention includes the isolated PNAs sequences according to SEQ ID NOs. 1, and 2. Another embodiment of the invention includes the production of PNAs sequences according to SEQ ID NOs. 1, and 2.

As noted, such compositions can also have antiviral therapeutic effects, and may be particularly effective at treating infection caused by SAR-CoV-2. At least one antisense FASTmers may be present in the composition at a pharmaceutically effective concentration or therapeutically effective amount. The pharmaceutically effective concentration of an antisense FASTmers will depend on several factors, including but not limited to the FASTmers's backbone composition, the affinity of the FASTmers for its target, the specificity of the FASTmers for its target, and the ability of the FASTmers to enter the cell. In certain embodiments, a pharmaceutically effective concentration of an antisense FASTmers is that concentration that prevents or treats COVID-19 infection in a subject in need thereof.

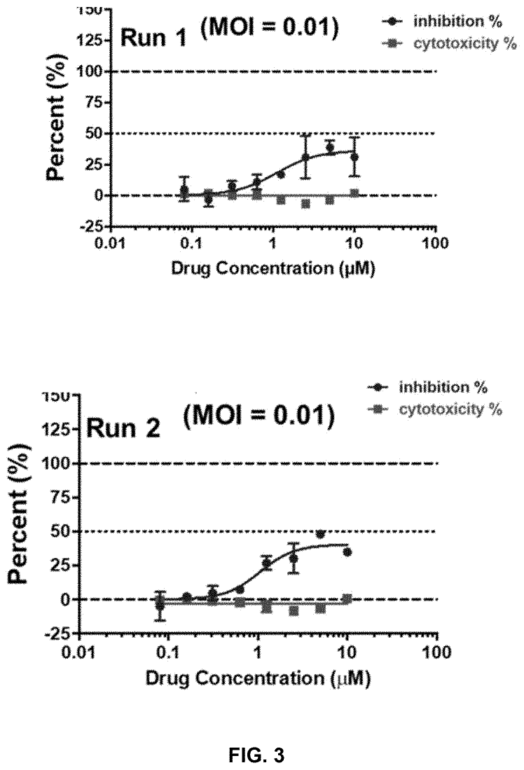

In certain embodiments, a FASTmer composition of the invention is a pharmaceutical composition. The compositions may routinely contain pharmaceutically acceptable concentrations of salt, buffering agents, preservatives and various compatible carriers or diluents. The proportion and identity of the pharmaceutically acceptable diluent is determined by chosen route of administration, compatibility with the antisense FASTmer oligomers of the composition, and standard pharmaceutical practice. Generally, the pharmaceutical composition will be formulated with components that will not significantly impair the biological activities of the antisense FASTmer oligomers. The pharmaceutical formulations of the present invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general, the compositions are prepared uniformly and intimately, bringing into association the active ingredients with liquid carriers or finely divided solid carriers or both, and then, if necessary, shaping the product. One of skill in the art will recognize that formulations are routinely designed according to their intended use, i.e., route of administration.

Wherein the composition comprises one or more antisense FASTmer oligomers having antiviral, and in particular anti-COVID-19 effects, a subject can be treated for a viral infection by administering an appropriate dose of the composition. Compositions described herein can be administered similarly to currently available antivirals or antibiotics, including but not limited to oral administration, nasal administration, intravenous administration, intramuscular administration, intraperitoneal administration, topical administration, local delivery methods, and in feed and water supplies. The formulation of therapeutic compositions and their subsequent administration (dosing) is believed to be within the skill of those in the art. Optimal dosing schedules can be calculated from measurements of drug accumulation in the body of a subject. Persons of ordinary skill can easily determine optimum dosages, dosing methodologies and repetition rates. Optimum dosages may vary depending on the relative potency of individual PNA FASTmers and can generally be estimated based on EC₅₀s found to be effective in in vitro and in vivo animal models as shown in FIGS. 3-5. In general, dosage is from 0.01 μg to 100 g per kg of body weight, and may be given once or more daily, weekly, or monthly. Persons of ordinary skill in the art can easily estimate repetition rates for dosing based on measured residence times and concentrations of the antisense oligomers in bodily fluids or tissues. Following successful treatment, it may be desirable to have the subject undergo maintenance therapy to prevent the recurrence of the disease state, wherein one or more PNAs is administered in maintenance doses, ranging from 0.01 μg to 100 g per kg of body weight. In certain embodiments, a patient is treated with a dosage of one or more PNAs that is at least about 1, at least about 2, at least about 3, at least about 4, at least about 5, at least about 6, at least about 7, at least about 8, at least about 9, at least about 10, at least about 15, at least about 20, at least about 25, at least about 30, at least about 35, at least about 40, at least about 45, at least about 50, at least about 60, at least about 70, at least about 80, at least about 90, or at least about 100 mg/kg body weight.

Another embodiment of the invention includes pharmaceutical compositions containing one or more antisense FASTmers configured to inhibit one or more host-derived miRNAs. In one preferred embodiment, the invention includes methods of treating COVID-19 in a subject in need thereof, comprising the step of administering a therapeutically effective amount of a pharmaceutical composition containing one or more antisense FASTmers configured to inhibit one or more host-derived miRNAs. As described above, the antisense FASTmers can be used in a synergistic combination with other known antiviral agents, and in particular anti-COVID-19 agents such as Remdesivir®, convalescent plasma, or one or more developmental vaccines directed towards SARS-CoV-2.

The inventive technology further comprises an antisense FASTmers, and preferably an antisense PNA FASTmer and the use of the composition for the preparation of a pharmaceutical composition, especially a therapeutic compound, e.g., for use in the prophylaxis or treatment of COVID-19 coronavirus infection. The inventive technology further describes methods of treatment or prevention of infections of COVID-19 coronavirus in subjects in need thereof using the composition comprising an antisense FASTmers, and preferably an antisense PNA FASTmer. Subjects to be treated for a COVID-19 infection can be selected from the group of: human; feed animals including but not limited to cattle, swine, poultry, goat, and sheep; companion animals, and laboratory animals.

Another embodiment of the invention includes pharmaceutical compositions containing one or more antisense PNA FASTmers configured to inhibit miRNA-2392. In one preferred embodiment, the invention includes methods of treating COVID-19 in a subject in need thereof, comprising the step of administering a therapeutically effective amount of a pharmaceutical compositions containing one or more antisense PNA FASTmers configured to inhibit miRNA-2392. Another embodiment of the invention includes the identification of novel miRNA targets for the treatment of viral infection, and in particular novel host-derived miRNA targets for the treatment of COVID-19. Another embodiment of the invention includes the rational design of novel miRNA inhibitors that may be used for the treatment of viral infection, and in particular novel inhibitors of host-derived miRNA for the treatment of COVID-19.

In one embodiment, at least one FASTmer, and preferably an antisense FASTmer configured to inhibit miRNA-2392 may be delivered to a host through a lipid nanoparticles. Preferably, lipid nanoparticles (LNPs) comprise: (a) at least one FASTmer, and preferably an antisense FASTmer configured to inhibit miRNA-2392, optionally comprised by the (pharmaceutical) composition as defined herein, (b) a cationic lipid, (c) optionally an aggregation reducing agent (such as polyethylene glycol (PEG) lipid or PEG-modified lipid), (d) optionally a non-cationic lipid (such as a neutral lipid), and (e) optionally, a sterol. In the context of the present invention, the term “lipid nanoparticle”, also referred to as “LNP”, is not restricted to any particular morphology, and includes any morphology generated when a cationic lipid and optionally one or more further lipids are combined, e.g., in an aqueous environment and/or in the presence of an antisense FASTmer. For example, a liposome, a lipid complex, a lipoplex, an emulsion, a micelle, a lipidic nanocapsule, a nanosuspension and the like are within the scope of a lipid nanoparticle (LNP). In some embodiments, LNPs comprise, in addition to the at least one FASTmer, and preferably an antisense FASTmer configured to inhibit miRNA-2392, optionally comprised by the (pharmaceutical) composition as defined herein, (i) at least one cationic lipid; (ii) a neutral lipid; (iii) a sterol, e.g., cholesterol; and (iv) a PEG-lipid, in a molar ratio of about 20-60% cationic lipid: 5-25% neutral lipid: 25-55% sterol; 0.5-15% PEG-lipid.

In some embodiments, the inventive FASTmer, and preferably an antisense FASTmer configured to inhibit miRNA-2392, optionally comprised by the (pharmaceutical) composition, may be formulated in an aminoalcohol lipidoid. Aminoalcohol lipidoids which may be used in the present invention may be prepared by the methods described in U.S. Pat. No. 8,450,298, herein incorporated by reference in its entirety. LNPs may include any cationic lipid suitable for forming a lipid nanoparticle. Preferably, the cationic lipid carries a net positive charge at about physiological pH. The cationic lipid may be an amino lipid. As used herein, the term “amino lipid” is meant to include those lipids having one or two fatty acid or fatty alkyl chains and an amino head group (including an alkylamino or dialkylamino group) that may be protonated to form a cationic lipid at physiological pH.

The cationic lipid may be, for example, N,N-dioleyl-N,N-dimethylammonium chloride (DODAC), N,N-distearyl-N,N-dimethylammonium bromide (DDAB), 1,2-dioleoyltrimethyl ammonium propane chloride (DOTAP) (also known as N-(2,3-dioleoyloxy)propyl)-N,N,N-trimethylammonium chloride and 1,2-Dioleyloxy-3-trimethylaminopropane chloride salt), N-(1-(2,3-dioleyloxy)propyl)-N,N,N-trimethylammonium chloride (DOTMA), N,N-dimethyl-2,3-dioleyloxy)propylamine (DODMA), 1,2-DiLinoleyloxy-N,N-dimethylaminopropane (DLinDMA), 1,2-Dilinolenyloxy-N,N-dimethylaminopropane (DLenDMA), 1,2-di-γ-linolenyloxy-N,N-dimethylaminopropane (γ-DLenDMA), 1,2-Dilinoleylcarbamoyloxy-3-dimethylaminopropane (DLin-C-DAP), 1,2-Dilinoleyoxy-3-(dimethylamino)acetoxypropane (DLin-DAC), 1,2-Dilinoleyoxy-3-morpholinopropane (DLin-MA), 1,2-Dilinoleoyl-3-dimethylaminopropane (DLinDAP), 1,2-Dilinoleylthio-3-dimethylaminopropane (DLin-S-DMA), 1-Linoleoyl-2-linoleyloxy-3-dimethylaminopropane (DLin-2-DMAP), 1,2-Dilinoleyloxy-3-trimethylaminopropane chloride salt (DLin-TMA.Ci), 1,2-Dilinoleoyi-3-trimethylaminopropane chloride salt (DLin-TAP.CI), 1,2-Dilinoleyloxy-3-(N-methylpiperazino)propane (DLin-MPZ), or 3-(N,N-Dilinoleylamino)-1,2-propanediol (DLinAP), 3-(N,N-Dioleylamino)-1,2-propanedio (DOAP), 1,2-Dilinoleyloxo-3-(2-N,N-dimethylamino)ethoxypropane (DLin-EG-DM A), 2,2-Dilinoleyl-4-dimethylaminomethyl-[1,3]-dioxolane (DLin-K-DMA) or analogs thereof, (3 aR, 5 s, 6aS)—N,N-dimethyl-2,2-di((9Z,12Z)-octadeca-9,12-dienyl)tetrahydro-3 aH-cyclopenta[d][1,3]dioxol-5-amine, (6Z,9Z,28Z,31Z)-heptatriaconta-6,9,28,31-tetraen-19-yl 4-(dimethylamino)butanoate (MC3), 1,1′-(2-(4-(2-((2-(bis(2-hydroxydodecyl)amino)ethyl)(2-hydroxydodecyl)amino)ethyl)piperazin-1-yl)ethylazanediyl)didodecan-2-ol (C12-200), 2,2-dilinoleyl-4-(2-dimethylaminoethyl)-[1,3]-dioxolane (DLin-K-C2-DMA), 2,2-dilinoleyl-4-dimethylaminomethyl-[1,3]-dioxolane (DLin-DMA), (6Z,9Z,28Z,31Z)-heptatriaconta-6,9,28,31-tetraen-19-yl 4-(dim ethyl amino) butanoate (DLin-M-C3-DMA), 3-((6Z,9Z,28Z,31Z)-heptatriaconta-6,9,28,31-tetraen-19-yloxy)-N,N-dimethylpropan-1-amine (MC3 Ether), 4-((6Z,9Z,28Z,31Z)-heptatriaconta-6,9,28,31-tetraen-19-yloxy)-N,N-dimethylbutan-1-amine (MC4 Ether), or any combination of any of the foregoing. Other cationic lipids include, but are not limited to, N,N-distearyl-N,N-dimethylammonium bromide (DDAB), 3P—(N—(N′,N′-dimethylaminoethane)-carbamoyl)cholesterol (DC-Choi), N-(1-(2,3-dioleyloxy)propyl)-N-2-(sperminecarboxamido)ethyl)-N,N-dimethylammonium trifluoracetate (DO SPA), dioctadecylamidoglycyl carboxyspermine (DOGS), 1,2-dileoyl-sn-3-phosphoethanolamine (DOPE), 1,2-dioleoyl-3-dimethylammonium propane (DODAP), N-(1,2-dimyristyloxyprop-3-yl)-N,N-dimethyl-N-hydroxyethyl ammonium bromide (DMRIE), and 2,2-Dilinoleyl-4-dimethylaminoethyl[1,3]-dioxolane (XTC). Additionally, commercial preparations of cationic lipids can be used, such as, e.g., LIPOFECTIN (including DOTMA and DOPE, available from GIBCO/BRL), and LIPOFECTAMINE (comprising DOSPA and DOPE, available from GIBCO/BRL). Other suitable (cationic) lipids are disclosed in WO2009/086558, WO2009/127060, WO2010/048536, WO2010/054406, WO2010/088537, WO2010/129709, WO2011/153493, US2011/0256175, US2012/0128760, US2012/0027803, and U.S. Pat. No. 8,158,601. In that context, the disclosures of WO2009/086558, WO2009/127060, WO2010/048536, WO2010/054406, WO2010/088537, WO2010/129709, WO2011/153493, US2011/0256175, US2012/0128760, US2012/0027803, and U.S. Pat. No. 8,158,601 are incorporated herewith by reference. In some embodiments the lipid may be selected from the group consisting of 98N12-5, C12-200, and ckk-E12.

The cationic lipid may also be an amino lipid. Suitable amino lipids include those having alternative fatty acid groups and other dialkylamino groups, including those in which the alkyl substituents are different (e.g., N-ethyl-N-methylamino-, and N-propyl-N-ethylamino-). In general, amino lipids having less saturated acyl chains are more easily sized, particularly when the complexes must be sized below about 0.3 microns, for purposes of filter sterilization. Amino lipids containing unsaturated fatty acids with carbon chain lengths in the range of C14 to C22 may be used. Other scaffolds can also be used to separate the amino group and the fatty acid or fatty alkyl portion of the amino lipid. Representative amino lipids include, but are not limited to, 1,2-dilinoleyoxy-3-(dimethylamino)acetoxypropane (DLin-DAC), 1,2-dilinoleyoxy-3 morpholinopropane (DLin-MA), 1,2-dilinoleoyl-3-dimethylaminopropane (DLinDAP), 1,2-dilinoleylthio-3-dimethylaminopropane (DLin-S-D A), 1-linoleoyl-2-linoleyloxy-3dimethylaminopropane (DLin-2-DMAP), 1,2-dilinoleyloxy-3-trimethylaminopropane chloride salt (DLin-TMA.CI), 1,2-dilinoleoyl-3-trimethylaminopropane chloride salt (DLin-TAP.CI), 1,2-dilinoleyloxy-3-(N-methylpiperazino)propane (DLin-MPZ), 3-(N,Ndilinoleylamino)-1,2-propanediol (DLinAP), 3-(N,N-dioleylamino)-1,2-propanediol (DOAP), 1,2-dilinoleyloxo-3-(2-N,N-dimethylamino)ethoxypropane (DLin-EG-DMA), and 2,2-dilinoleyl-4-dimethylaminomethyl-[1,3]-dioxolane (DLin-K-DMA), 2,2-dilinoleyl-4-(2-dimethylaminoethyl)-[1,3]-dioxolane (DLin-KC2-DMA); dilinoleyl-methyl-4-dimethylaminobutyrate (DLin-MC3-DMA); C3 (US20100324120).

In some embodiments, amino or cationic lipids have at least one protonatable or deprotonatable group, such that the lipid is positively charged at a pH at or below physiological pH (e.g. pH 7.4), and neutral at a second pH, preferably at or above physiological pH. It will, of course, be understood that the addition or removal of protons as a function of pH is an equilibrium process, and that the reference to a charged or a neutral lipid refers to the nature of the predominant species and does not require that all of the lipid be present in the charged or neutral form. Lipids that have more than one protonatable or deprotonatable group, or which are zwitterionic, are not excluded from use in the invention. In some embodiments, the protonatable lipids have a pKa of the protonatable group in the range of about 4 to about 11, e.g., a pKa of about 5 to about 7. LNPs can include two or more cationic lipids. The cationic lipids can be selected to contribute different advantageous properties. For example, cationic lipids that differ in properties such as amine pKa, chemical stability, half-life in circulation, half-life in tissue, net accumulation in tissue, or toxicity can be used in the LNP. In particular, the cationic lipids can be chosen so that the properties of the mixed-LNP are more desirable than the properties of a single-LNP of individual lipids.

In some embodiments, the cationic lipid is present in a ratio of from about 20 mol % to about 70 or 75 mol % or from about 45 to about 65 mol % or about 20, 25, 30, 35, 40, 45, 50, 55, 60, 65, or about 70 mol % of the total lipid present in the LNP. In further embodiments, the LNPs comprise from about 25% to about 75% on a molar basis of cationic lipid, e.g., from about 20 to about 70%, from about 35 to about 65%, from about 45 to about 65%, about 60%, about 57.5%, about 57.1%, about 50% or about 40% on a molar basis (based upon 100% total moles of lipid in the lipid nanoparticle). In some embodiments, the ratio of cationic lipid to nucleic acid is from about 3 to about 15, such as from about 5 to about 13 or from about 7 to about 11. The amount of the permanently cationic lipid or lipidoid may be selected taking the amount of the nucleic acid cargo into account. In one embodiment, these amounts are selected such as to result in an N/P ratio of the nanoparticle(s) or of the composition in the range from about 0.1 to about 20. In this context, the N/P ratio is defined as the mole ratio of the nitrogen atoms (“N”) of the basic nitrogen-containing groups of the lipid or lipidoid to the phosphate groups (“P”) of the antisense FASTmer which is used as cargo. The N/P ratio may be calculated on the basis that, for example, Ipg RNA typically contains about 3 nmol phosphate residues, provided that the antisense FASTmer exhibits a statistical distribution of bases. The “N”-value of the lipid or lipidoid may be calculated on the basis of its molecular weight and the relative content of permanently cationic and—if present—cationisable groups. In certain embodiments, the LNP comprises one or more additional lipids which stabilize the formation of particles during their formation.

In some embodiments, non-cationic may be used. The non-cationic lipid can be a neutral lipid, an anionic lipid, or an amphipathic lipid. Neutral lipids, when present, can be any of a number of lipid species which exist either in an uncharged or neutral zwitterionic form at physiological pH. Such lipids include, for example, diacylphosphatidylcholine, diacylphosphatidylethanolamine, ceramide, sphingomyelin, dihydrosphingomyelin, cephalin, and cerebrosides. The selection of neutral lipids for use in the particles described herein is generally guided by consideration of, e.g., LNP size and stability of the LNP in the bloodstream. Preferably, the neutral lipid is a lipid having two acyl groups (e.g., diacylphosphatidylcholine and diacylphosphatidylethanolamine). In some embodiments, the neutral lipids contain saturated fatty acids with carbon chain lengths in the range of C₁₀ to C₂₀. In other embodiments, neutral lipids with mono or diunsaturated fatty acids with carbon chain lengths in the range of CIO to C20 are used. Additionally, neutral lipids having mixtures of saturated and unsaturated fatty acid chains can be used. Suitable neutral lipids include, but are not limited to, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoylphosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), dimyristoyl phosphatidylcholine (DMPC), distearoyl-phosphatidyl-ethanolamine (DSPE), SM, 16-0-monomethyl PE, 16-0-dimethyl PE, 18-1-trans PE, 1-stearoyl-2-oleoyl-phosphatidyethanolamine (SOPE), cholesterol, or a mixture thereof. Anionic lipids suitable for use in LNPs include, but are not limited to, phosphatidylglycerol, cardiolipin, diacylphosphatidylserine, diacylphosphatidic acid, N-dodecanoyl phosphatidylethanoloamine, N-succinyl phosphatidylethanolamine, N-glutaryl phosphatidylethanolamine, lysylphosphatidylglycerol, and other anionic modifying groups joined to neutral lipids. In one embodiment, the neutral lipid is 1,2-distearoyl-sn-glycero-3phosphocholine (DSPC).

In some embodiments, the LNPs comprise a neutral lipid selected from DSPC, DPPC, DMPC, DOPC, POPC, DOPE and SM. In various embodiments, the molar ratio of the cationic lipid to the neutral lipid ranges from about 2:1 to about 8:1. Amphipathic lipids refer to any suitable material, wherein the hydrophobic portion of the lipid material orients into a hydrophobic phase, while the hydrophilic portion orients toward the aqueous phase. Such compounds include, but are not limited to, phospholipids, aminolipids, and sphingolipids. Representative phospholipids include sphingomyelin, phosphatidylcholine, phosphatidylethanolamine, phosphatidylserine, phosphatidylinositol, phosphatidic acid, paimitoyloleoyl phosphatdylcholine, \hat{ }phosphatidylcholine, lysophosphatidylethanolamine, dipalmitoylphosphatidylcholine, dioleoylphosphatidylcholine, di stearoylphosphatidylcholine, or dilinoleoylphosphatidylcholine. Other phosphorus-lacking compounds, such as sphingolipids, glycosphingolipid families, diacylglycerols, and beta-acyloxyacids, can also be used.

In some embodiments, the non-cationic lipid is present in a ratio of from about 5 mol % to about 90 mol %, about 5 mol % to about 10 mol %, about 5, 10, 15, 20, 25, 30, 35, 40, 45, 50, 55, 60, 65, 70, 75, 80, 85, or about 90 mol % of the total lipid present in the LNP. In some embodiments, LNPs comprise from about 0% to about 15 or 45% on a molar basis of neutral lipid, e.g., from about 3 to about 12% or from about 5 to about 10%. For instance, LNPs may include about 15%, about 10%, about 7.5%, or about 7.1% of neutral lipid on a molar basis (based upon 100% total moles of lipid in the LNP).

In some embodiments, a sterol may be used. The sterol is preferably cholesterol. The sterol can be present in a ratio of about 10 mol % to about 60 mol % or about 25 mol % to about 40 mol % of the LNP. In some embodiments, the sterol is present in a ratio of about 10, 15, 20, 25, 30, 35, 40, 45, 50, 55, or about 60 mol % of the total lipid present in the LNP. In other embodiments, LNPs comprise from about 5% to about 50% on a molar basis of the sterol, e.g., about 15% to about 45%, about 20% to about 40%, about 48%, about 40%, about 38.5%, about 35%, about 34.4%, about 31.5% or about 31% on a molar basis (based upon 100% total moles of lipid in the LNP). In some embodiments, an aggregation reducing agent may be employed. The aggregation reducing agent can be a lipid capable of reducing aggregation. Examples of such lipids include, but are not limited to, polyethylene glycol (PEG)-modified lipids, monosialoganglioside Gml, and polyamide oligomers (PAO) such as those described in U.S. Pat. No. 6,320,017, which is incorporated by reference in its entirety. Other compounds with uncharged, hydrophilic, steric-barrier moieties, which prevent aggregation during formulation, like PEG, Gml or ATTA, can also be coupled to lipids. ATTA-lipids are described, e.g., in U.S. Pat. No. 6,320,017, and PEG-lipid conjugates are described, e.g., in U.S. Pat. Nos. 5,820,873, 5,534,499, 5,885,613, US20150376115A1 and WO2015/199952, each of which is incorporated by reference in its entirety.

The aggregation reducing agent may be, for example, selected from a polyethyleneglycol (PEG)-lipid including, without limitation, a PEG-diacylglycerol (DAG), a PEG-dialkylglycerol, a PEG-dialkyloxypropyl (DAA), a PEG-phospholipid, a PEG-ceramide (Cer), or a mixture thereof (such as PEG-Cer14 or PEG-Cer20). The PEG-DAA conjugate may be, for example, a PEG-dilauryloxypropyl (C12), a PEG-dimyristyloxypropyl (C14), a PEG-dipalmityloxypropyl (C16), or a PEG-distearyloxypropyl (C18). Other pegylated-lipids include, but are not limited to, polyethylene glycol-didimyristoyl glycerol (C14-PEG or PEG-C14, where PEG has an average molecular weight of 2000 Da) (PEG-DMG); (R)-2,3-bis(octadecyloxy)propyl-1-(methoxy polyethylene glycol)2000)propylcarbamate) (PEG-DSG); PEG-carbamoyl-1,2-dimyristyloxypropylamine, in which PEG has an average molecular weight of 2000 Da (PEG-cDMA); N-Acetylgalactosamine-((R)-2,3-bis(octadecyloxy)propyl-1-(methoxy polyethylene glycol)2000)propylcarbamate)) (GalNAc-PEG-DSG); mPEG (mw2000)-diastearoylphosphatidyl-ethanolamine (PEG-DSPE); and polyethylene glycol-dipalmitoylglycerol (PEG-DPG). In some embodiments, the aggregation reducing agent is PEG-DMG. In other embodiments, the aggregation reducing agent is PEG-c-DMA.

In various embodiments, the molar ratio of the cationic lipid to the PEGylated lipid ranges from about 100:1 to about 25:1. In a preferred embodiment, the composition of LNPs may be influenced by, inter alia, the selection of the cationic lipid component, the degree of cationic lipid saturation, the nature of the PEGylation, the ratio of all components and biophysical parameters such as its size. In one example by Semple et al. (Semple et al. Nature Biotech. 201028: 172-176; herein incorporated by reference in its entirety), the LNP composition was composed of 57.1% cationic lipid, 7.1% dipalmitoylphosphatidylcholine, 34.3% cholesterol, and 1.4% PEG-c-DMA (Basha et al. Mol Ther. 2011 19:2186-2200; herein incorporated by reference in its entirety). In some embodiments, LNPs may comprise from about 35 to about 45% cationic lipid, from about 40% to about 50% cationic lipid, from about 50% to about 60% cationic lipid and/or from about 55% to about 65% cationic lipid. In some embodiments, the ratio of lipid to FASTmer may range from about 5:1 to about 20:1, from about 10:1 to about 25:1, from about 15:1 to about 30:1 and/or at least 30:1. The average molecular weight of the PEG moiety in the PEG-modified lipids can range from about 500 to about 8,000 Daltons (e.g., from about 1,000 to about 4,000 Daltons). In one preferred embodiment, the average molecular weight of the PEG moiety is about 2,000 Daltons.

The concentration of the aggregation reducing agent may range from about 0.1 to about 15 mol %, per 100% total moles of lipid in the LNP. In some embodiments, LNPs include less than about 3, 2, or 1 mole percent of PEG or PEG-modified lipid, based on the total moles of lipid in the LNP. In further embodiments, LNPs comprise from about 0.1% to about 20% of the PEG-modified lipid on a molar basis, e.g., about 0.5 to about 10%, about 0.5 to about 5%, about 10%, about 5%, about 3.5%, about 3%, about 2,5%, about 2%, about 1.5%, about 1%, about 0.5%, or about 0.3% on a molar basis (based on 100% total moles of lipids in the LNP). Different LNPs having varied molar ratios of cationic lipid, non-cationic (or neutral) lipid, sterol (e.g., cholesterol), and aggregation reducing agent (such as a PEG-modified lipid) on a molar basis (based upon the total moles of lipid in the lipid nanoparticles).

The total amount of nucleic acid, particularly the one or more antisense FASTmers in the lipid nanoparticles varies and may be defined depending on the e.g., an antisense FASTmer to total lipid w/w ratio. In one embodiment of the invention the antisense FASTmer to total lipid ratio is less than 0.06 w/w, preferably between 0.03 w/w and 0.04 w/w.

In some embodiments, LNPs occur as liposomes or lipoplexes as described in further detail below. In some embodiments, LNPs have a median diameter size of from about 50 nm to about 300 nm, such as from about 50 nm to about 250 nm, for example, from about 50 nm to about 200 nm. In some embodiments, smaller LNPs may be used. Such particles may comprise a diameter from below 0.1 pm up to 100 nm such as, but not limited to, less than 0.1 pm, less than 1.0 pm, less than 5 pm, less than 10 pm, less than 15 pm, less than 20 pm, less than 25 pm, less than 30 pm, less than 35 pm, less than 40 pm, less than 50 pm, less than 55 pm, less than 60 pm, less than 65 pm, less than 70 pm, less than 75 pm, less than 80 pm, less than 85 pm, less than 90 pm, less than 95 pm, less than 100 pm, less than 125 pm, less than 150 pm, less than 175 pm, less than 200 pm, less than 225 pm, less than 250 pm, less than 275 pm, less than 300 pm, less than 325 pm, less than 350 pm, less than 375 pm, less than 400 pm, less than 425 pm, less than 450 pm, less than 475 pm, less than 500 pm, less than 525 pm, less than 550 pm, less than 575 pm, less than 600 pm, less than 625 pm, less than 650 pm, less than 675 pm, less than 700 pm, less than 725 pm, less than 750 pm, less than 775 pm, less than 800 pm, less than 825 pm, less than 850 pm, less than 875 pm, less than 900 pm, less than 925 pm, less than 950 pm, less than 975 pm, In another embodiment, nucleic acids may be delivered using smaller LNPs which may comprise a diameter from about 1 nm to about 100 nm, from about 1 nm to about 10 nm, about 1 nm to about 20 nm, from about 1 nm to about 30 nm, from about 1 nm to about 40 nm, from about 1 nm to about 50 nm, from about 1 nm to about 60 nm, from about 1 nm to about 70 nm, from about 1 nm to about 80 nm, from about 1 nm to about 90 nm, from about 5 nm to about from 100 nm, from about 5 nm to about 10 nm, about 5 nm to about 20 nm, from about 5 nm to about 30 nm, from about 5 nm to about 40 nm, from about 5 nm to about 50 nm, from about 5 nm to about 60 nm, from about 5 nm to about 70 nm, from about 5 nm to about 80 nm, from about 5 nm to about 90 nm, about 10 to about 50 nm, from about 20 to about 50 nm, from about 30 to about 50 nm, from about 40 to about 50 nm, from about 20 to about 60 nm, from about 30 to about 60 nm, from about 40 to about 60 nm, from about 20 to about 70 nm, from about 30 to about 70 nm, from about 40 to about 70 nm, from about 50 to about 70 nm, from about 60 to about 70 nm, from about 20 to about 80 nm, from about 30 to about 80 nm, from about 40 to about 80 nm, from about 50 to about 80 nm, from about 60 to about 80 nm, from about 20 to about 90 nm, from about 30 to about 90 nm, from about 40 to about 90 nm, from about 50 to about 90 nm, from about 60 to about 90 nm and/or from about 70 to about 90 nm. In some embodiments, the LNP may have a diameter greater than 100 nm, greater than 150 nm, greater than 200 nm, greater than 250 nm, greater than 300 nm, greater than 350 nm, greater than 400 nm, greater than 450 nm, greater than 500 nm, greater than 550 nm, greater than 600 nm, greater than 650 nm, greater than 700 nm, greater than 750 nm, greater than 800 nm, greater than 850 nm, greater than 900 nm, greater than 950 nm or greater than 1000 nm.

In other embodiments, LNPs have a single mode particle size distribution (i.e., they are not bi- or poly-modal). LNPs, as used herein may further comprise one or more lipids and/or other components in addition to those mentioned above.

Other lipids may be included in the liposome compositions for a variety of purposes, such as to prevent lipid oxidation or to attach ligands onto the liposome surface. Any of a number of lipids may be present in LNPs, including amphipathic, neutral, cationic, and anionic lipids. Such lipids can be used alone or in combination. Additional components that may be present in an LNP include bilayer stabilizing components such as polyamide oligomers (see, e.g., U.S. Pat. No. 6,320,017, which is incorporated by reference in its entirety), peptides, proteins, and detergents.

In some embodiments, the inventive FASTmers, optionally comprised by (pharmaceutical) compositions are formulated as liposomes. Cationic lipid-based liposomes are able to complex with negatively charged nucleic acids (e.g., FASTmers) via electrostatic interactions, resulting in complexes that offer biocompatibility, low toxicity, and the possibility of the large-scale production required for in vivo clinical applications. Liposomes can fuse with the plasma membrane for uptake; once inside the cell, the liposomes are processed via the endocytic pathway and the nucleic acid is then released from the endosome/carrier into the cytoplasm. Liposomes have long been perceived as drug delivery vehicles because of their superior biocompatibility, given that liposomes are basically analogs of biological membranes, and can be prepared from both natural and synthetic phospholipids (Int J Nanomedicine. 2014; 9: 1833-1843).

Liposomes typically consist of a lipid bilayer that can be composed of cationic, anionic, or neutral (phospho)lipids and cholesterol, which encloses an aqueous core. Both the lipid bilayer and the aqueous space can incorporate hydrophobic or hydrophilic compounds, respectively. Liposomes may have one or more lipid membranes. Liposomes can be single-layered, referred to as unilamellar, or multi-layered, referred to as multilamellar. Liposome characteristics and behavior in vivo can be modified by addition of a hydrophilic polymer coating, e.g., polyethylene glycol (PEG), to the liposome surface to confer steric stabilization. Furthermore, liposomes can be used for specific targeting by attaching ligands (e.g., antibodies, peptides, and carbohydrates) to its surface or to the terminal end of the attached PEG chains (Front Pharmacol. 2015 Dec. 1; 6:286). Liposomes are typically present as spherical vesicles and can range in size from 20 nm to a few microns. Liposomes can be of different sizes such as, but not limited to, a multilamellar vesicle (MLV) which may be hundreds of nanometers in diameter and may contain a series of concentric bilayers separated by narrow aqueous compartments, a small unicellular vesicle (SUV) which may be smaller than 50 nm in diameter, and a large unilamellar vesicle (LUV) which may be between 50 and 500 nm in diameter. Liposome design may include, but is not limited to, opsonins or ligands in order to improve the attachment of liposomes to unhealthy tissue or to activate events such as, but not limited to, endocytosis. Liposomes may contain a low or a high pH in order to improve the delivery of the pharmaceutical formulations.

As a non-limiting example, liposomes such as synthetic membrane vesicles may be prepared by the methods, apparatus and devices described in US Patent Publication No. US20130177638, US20130177637, US20130177636, US20130177635, US20130177634, US20130177633, US20130183375, US20130183373 and US20130183372, the contents of each of which are herein incorporated by reference in its entirety. The inventive FASTmer, optionally comprised by the (pharmaceutical) composition, may be encapsulated by the liposome and/or it may be contained in an aqueous core which may then be encapsulated by the liposome (see International Pub. Nos. WO2012/031046, WO2012/031043, WO2012/030901 and WO2012/006378 and US Patent Publication No. US20130189351, US20130195969 and US20130202684; the contents of each of which are herein incorporated by reference in their entirety).

In some embodiments, the inventive FASTmer, and preferably an antisense FASTmer configured to inhibit miRNA-2392, optionally comprised by the (pharmaceutical) composition, may be formulated in liposomes such as, but not limited to, DiLa2 liposomes (Marina Biotech, Bothell, Wash.), SMARTICLES® (Marina Biotech, Bothell, Wash.), neutral DOPC (1,2-dioleoyl-sn-glycero-3-phosphocholine) based liposomes (e.g., siRNA delivery for (Landen et al. Cancer Biology & Therapy 2006 5(12)1708-1713); herein incorporated by reference in its entirety) and hyaluronan-coated liposomes (Quiet Therapeutics, Israel).

In some embodiments, the inventive FASTmer, and preferably an antisense FASTmer configured to inhibit miRNA-2392, optionally comprised by the (pharmaceutical) composition, is formulated in the form of lipoplexes, i.e., cationic lipid bilayers sandwiched between nucleic acid (e.g. FASTmer) layers. Cationic lipids, such as DOTAP, (1,2-dioleoyl-3-trimethylammonium-propane) and DOTMA (N-[1-(2,3-dioleoyloxy)propyl]-N,N,N-trimethyl-ammonium methyl sulfate) can form complexes or lipoplexes with negatively charged nucleic acids to form nanoparticles by electrostatic interaction, providing high in vitro transfection efficiency.

In some embodiments, the inventive FASTmer, and preferably an antisense FASTmer configured to inhibit miRNA-2392, optionally comprised by the (pharmaceutical) composition as defined herein, is formulated in the form of nanoliposomes, preferably neutral lipid-based nanoliposomes such as 1,2-dioleoyl-sn-glycero-3-phosphatidylcholine (DOPC)-based nanoliposomes (Adv Drug Deliv Rev. 2014 February; 66: 110-116.). In some embodiments, the inventive FASTmer, and preferably an antisense FASTmer configured to inhibit miRNA-2392, optionally comprised by the (pharmaceutical) composition as defined herein, is provided in the form of an emulsion. In some embodiment, said FASTmer is formulated in a cationic oil-in-water emulsion, wherein the emulsion particle comprises an oil core and a cationic lipid which can interact with said FASTmer, anchoring the molecule to the emulsion particle (see International Pub. No. WO2012/006380; herein incorporated by reference in its entirety). In some embodiments, said FASTmer is formulated in a water-in-oil emulsion comprising a continuous hydrophobic phase in which the hydrophilic phase is dispersed. As a non-limiting example, the emulsion may be made by the methods described in International Publication No. WO2010/87791, the contents of which are herein incorporated by reference in its entirety.

The invention now being generally described will be more readily understood by reference to the following examples, which are included merely for the purposes of illustration of certain aspects of the embodiments of the present invention. The examples are not intended to limit the invention, as one of skill in the art would recognize from the above teachings and the following examples that other techniques and methods can satisfy the claims and can be employed without departing from the scope of the claimed invention. Indeed, while this invention has been particularly shown and described with references to preferred embodiments thereof, it will be understood by those skilled in the art that various changes in form and details may be made therein without departing from the scope of the invention encompassed by the appended claims.

### EXAMPLES

### Example 1: Identification of miRNA Interactions in COVID-19 Infected Host

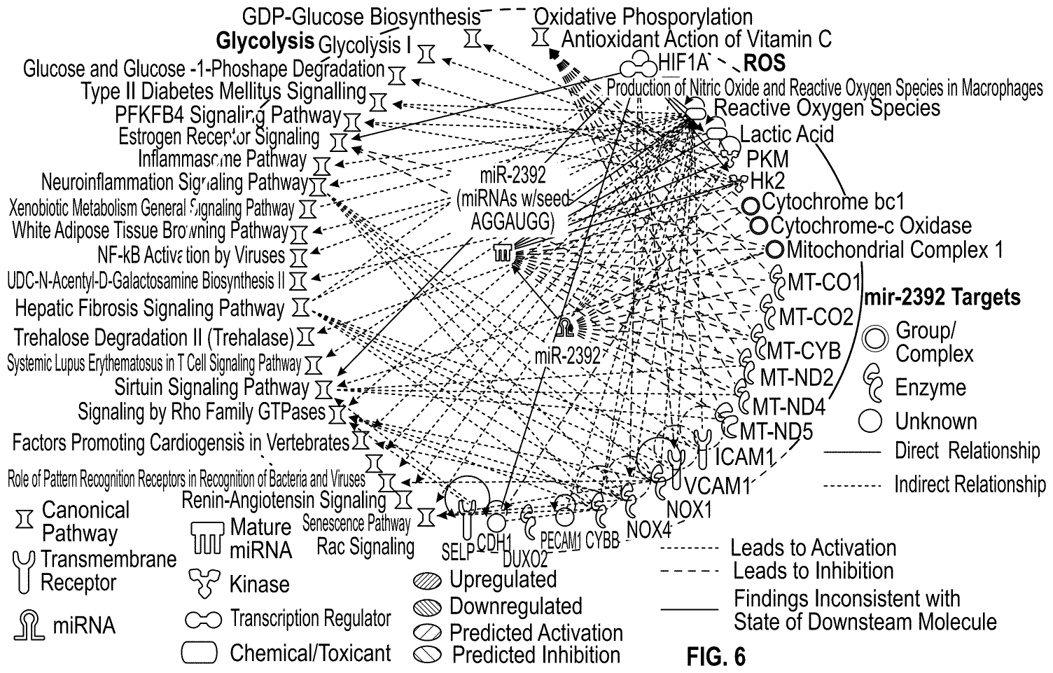

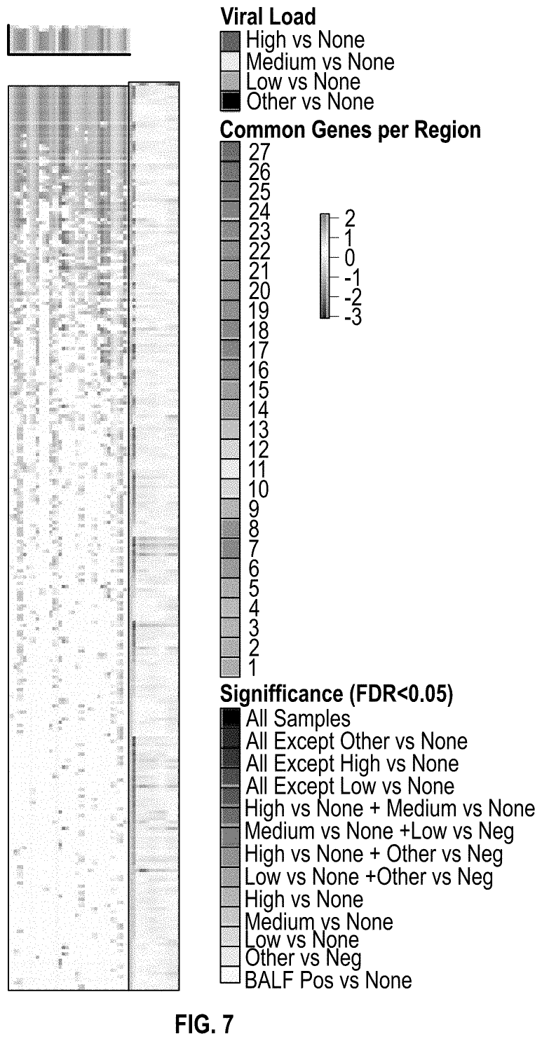

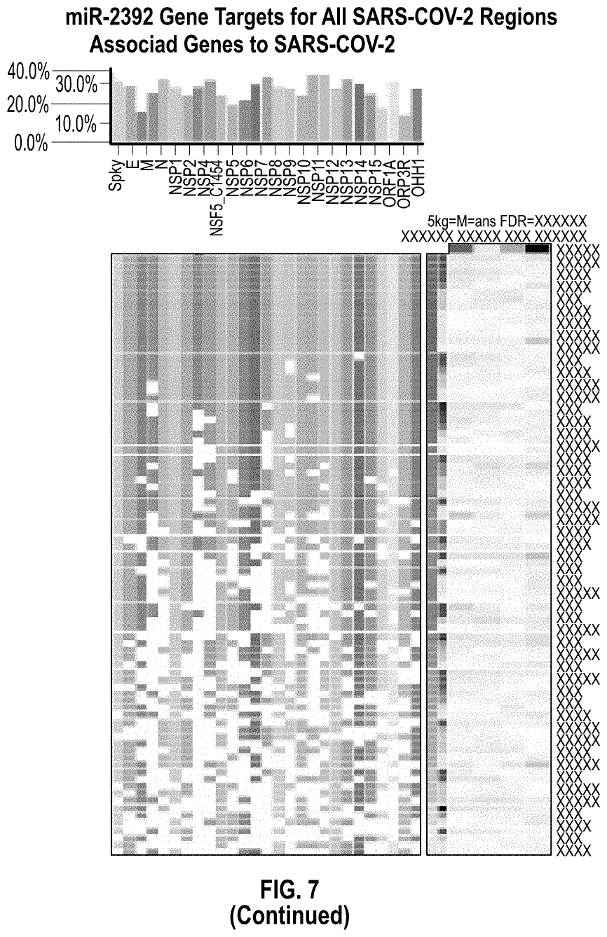

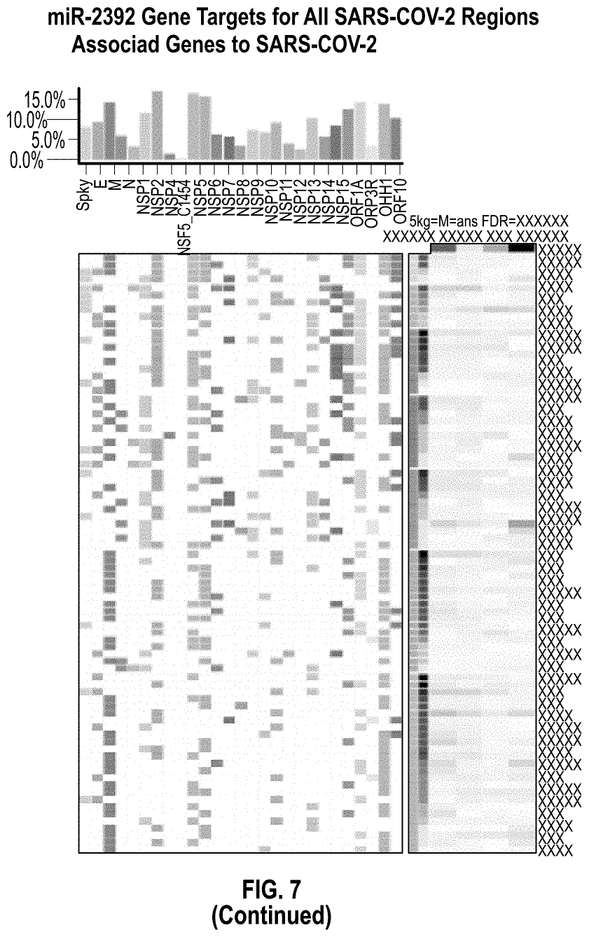

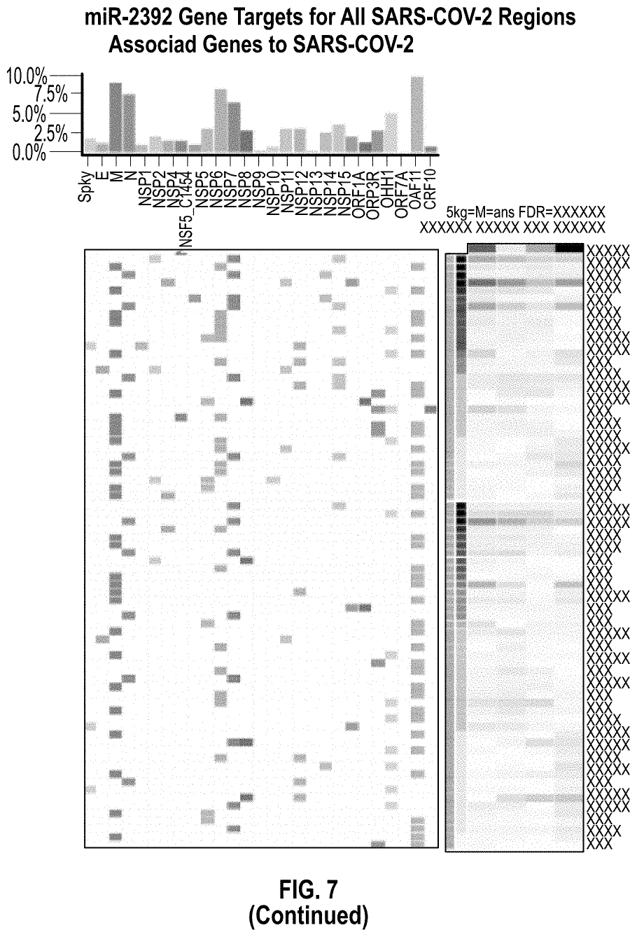

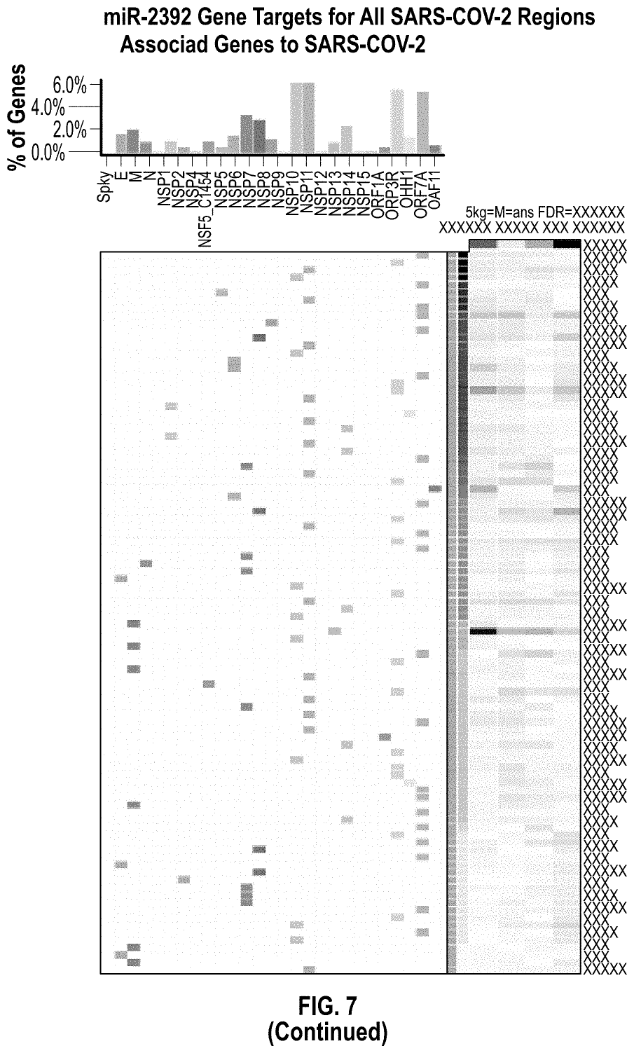

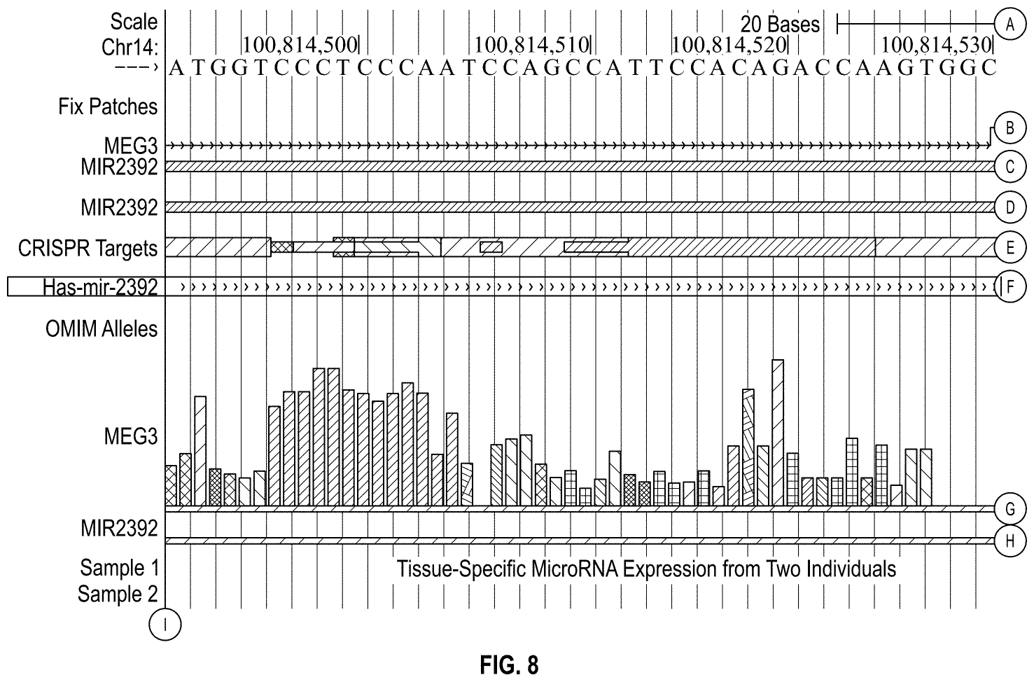

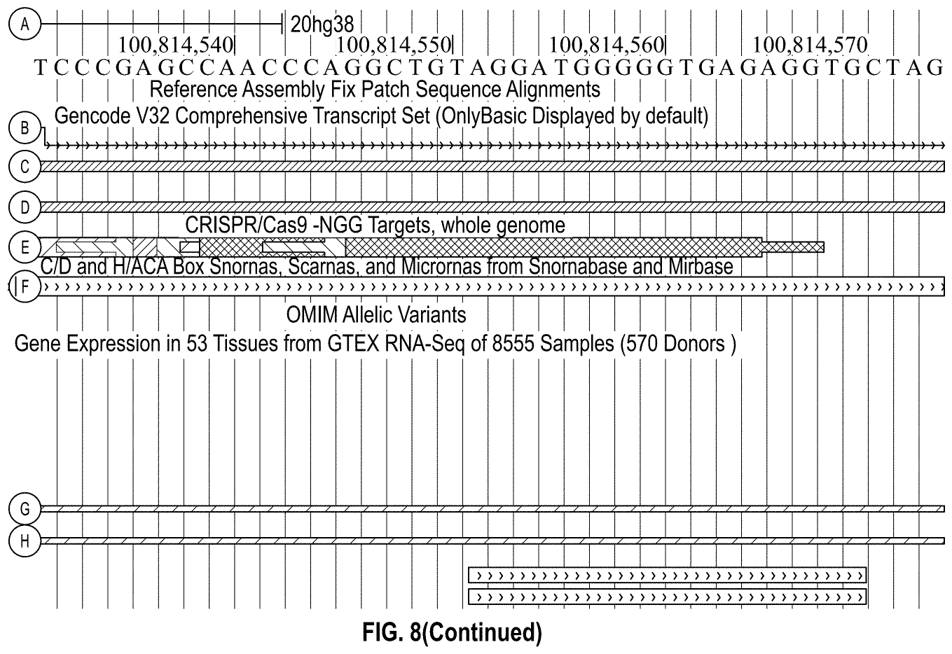

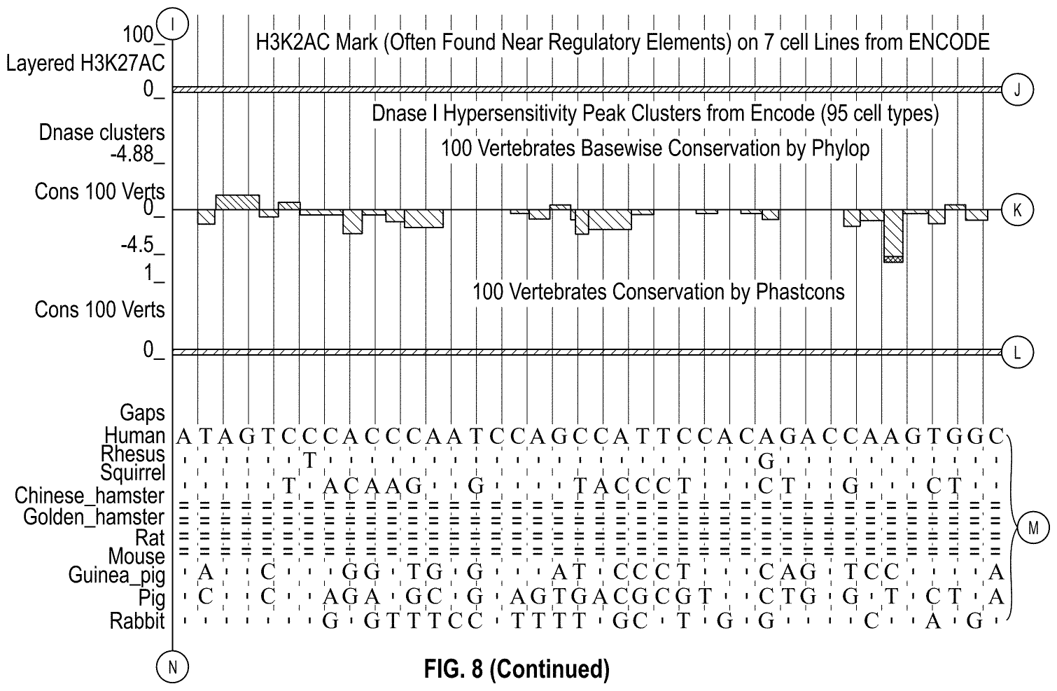

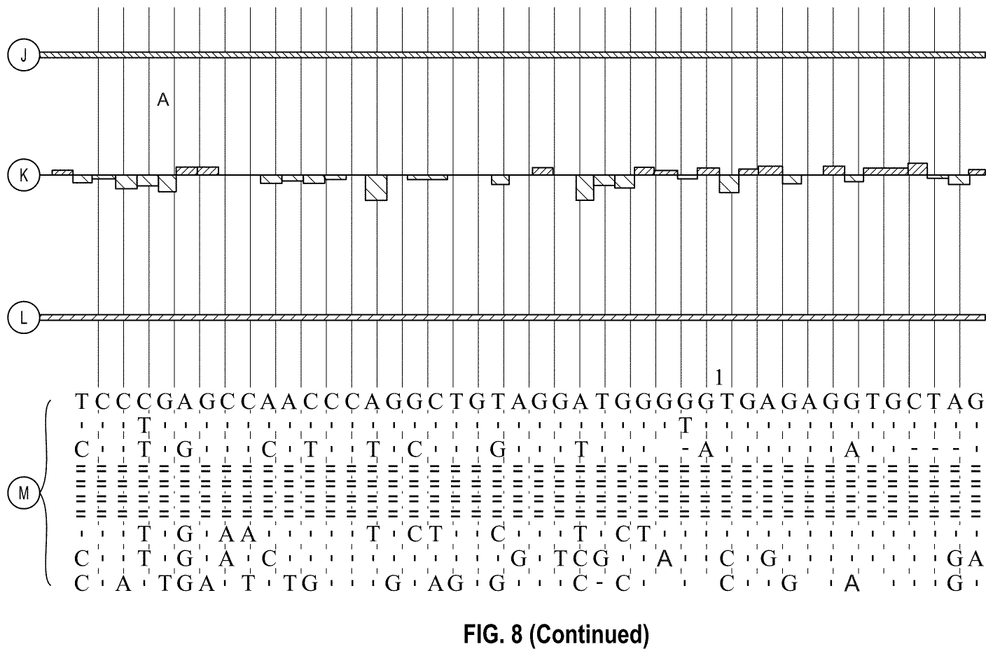

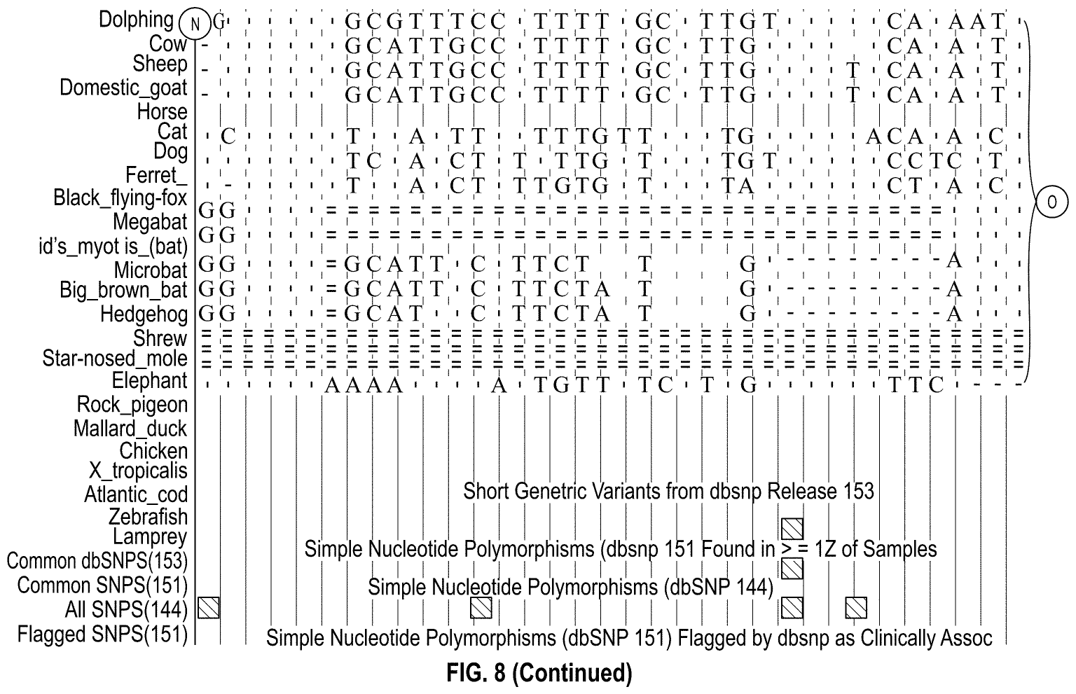

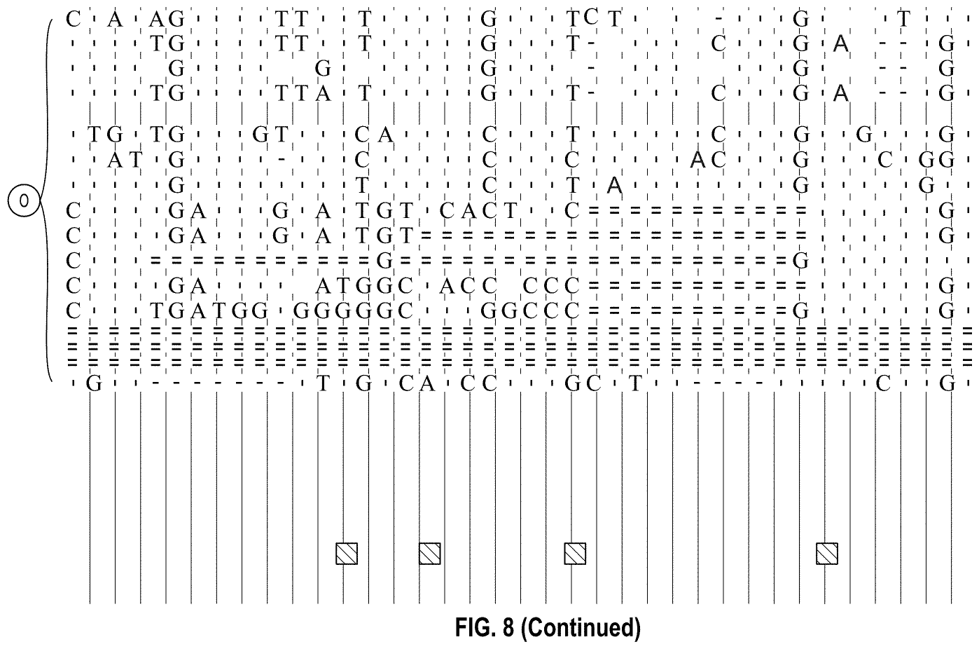

In inventive technology described herein includes the development of a miRNA based therapeutic/vaccine to treat COVID-19. In one embodiment, the present inventors identified host-derived miRNA targets to be inhibited through the use of the rationally designed inhibitor. More particularly, the present invention identified specific miRNA signatures associated with COVID-19 infection in a subject and identified within that signature potential miRNAs that could be targeted for inhibition using rationally designed antisense oligomer inhibitors, generally described herein as FASTmers. As identified in FIG. 6, the miRNA signature for COVID-19 was identified as the following: miRNA-181a-5p, miRNA-10, miRNA-10a-5p, miRNA-2393, miRNA-1-5p, miRNA-34a-5, miRNA-30c-5p, miRNA-29b-3p, miRNA-155-5p, and miRNA-124-3p. As again shown in FIG. 6, the host-derived miRNA-2392 has been shown to be a key miRNA involved in facilitating COVID-19 infection. As shown in FIG. 7, using RNA-sequencing data from multiple patient sample locations the present inventors determined and predicted miRNAs that are commonly being expressed in the COVID-19 positive patients compared to negative tested patients. From this data a list of initial miRNAs targets were identified and a series of rationally designed antagomirs were developed to inhibit the specific miRNAs.

Notably, the present inventors have identified miRNA-2392 as prime candidate driving COVID-19 infection. As demonstrated in FIG. 6, miRNA-2392 suppresses mitochondrial functions and is highly involved with both the host response to the viral infection and also has direct impact on the virus itself. Further validation on the impact of the miRNA-2392 on the host during COVID-19 infection was done by utilizing RNA-sequencing data from nasal pharyngeal swabs on COVID-19 positive and negative patients. Analysis of this RNA-seq data_showed that downstream miRNA-2392 gene targets are heavily involved as a function of viral load during COVID-19 infections.

### Example 2: Identification of Highly Conserved Nature of miRNA-2392

As generally shown in FIG. 7, the present inventors demonstrated that miRNA-2392 is highly conserved which accounts for the easily transmission of this virus across different species. For example, it is known that COVID-19 does not impact mice which is confirmed as the data shows that miRNA-2392 is not conserved in mice.

The present inventors further identified that miRNA-2392 has an 8 bp overlap target sequence that can be found in the SARS-COV-2 virus. This occurs in key regions of the virus, with one being in the Spike Protein region, which is known to be a major antigenic determinant of SARS-Cov-2 which is a type I viral fusion protein that binds to ACE2 via its receptor binding domain (RBD) to gain cell entry.

### Example 3: Application of FAST Platform to Rationally Design Antisense miRNA-2392 Inhibitor

Having discovered the role of miRNA-2392 in driving COVID-19 infection, and further identified the highly conserved region of miRNA-2392, the present inventors next sought to rationally design an antisense miRNA-2392 inhibitor, or FASTmer as generally referred to herein.

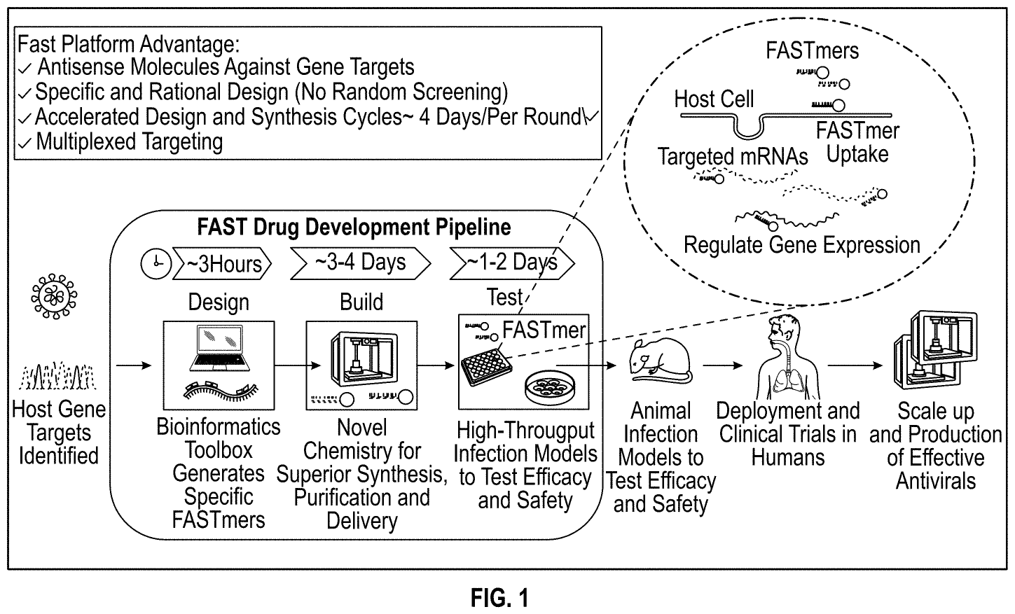

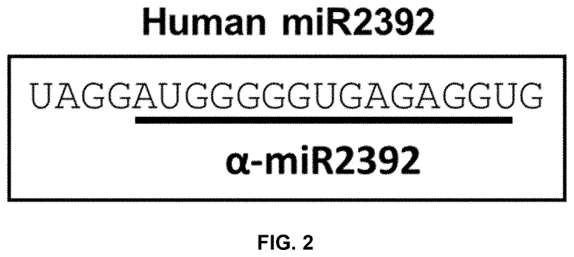

As outlined in FIG. 1, the FAST platform (generally described in PCT/US2020/045638, the figures, specification and claims being specifically incorporated herein by reference) was used to complete the rational design, build, and testing steps of an antisense FASTmer that targets miRNA-2392 within less than a single week. First, the PNA Finder toolbox was used to find potential antisense targets in the following regions of the human genome (GRCh38.p12, Genbank: GCA 000001405.27): miRNA-2392. Multiple 15-mer PNA candidates were derived from each region of the genome, and were filtered for high predicted aqueous solubility, according to solubility metrics known in the field, as well as a lack of self-complementing sequences.

As shown in Table 1 below, the resulting FASTmer α-miR2392 was designed to bind complementarily to the human miRNA-2392. The 20 nucleotide miRNA sequence yields six possible 15mer FASTmer moieties that may bind to the sequence and inhibit binding to a target as a regulatory agent. The six sequences were screened for solubility as well as viral and human off-targets, as described above. The selected PNA was found to have no 2-mismatch viral alignments, no zero-mismatch human off-targets, and 23 one-mismatch human off-targets.

Two versions of the FASTmer α-miR2392 were designed to bind complementarily to the human miRNA 2392. The 20 nucleotide miRNA sequence yields six possible 15mer PNA sequences that are predicted to bind to the miRNA and prevent its binding to a target. This is expected to inhibit its function as a regulatory agent. The six sequences were screened for solubility as well as viral and human off-targets, ranging from zero to two allowed off-targets in the alignment search. The selected PNAs (α-miR2392 v1 and v2) were found to have no 2-mismatch viral alignments. The PNA α-miR2392 v1 (SEQ ID NO. 1) has no zero-mismatch human off-targets, whereas α-miR2392 v2 (SEQ ID NO. 2) shows two zero-mismatch off-targets. The two PNA have 23 and 53 one-mismatch human off-targets, respectively. The PNA were synthesized using Fmoc chemistry and purified. They were conjugated with nanoparticles, as these have been demonstrated in previous work to improve transport in mammalian systems.

### Example 4: Inhibition of miRNA-2392 by Rationally Designed FASTmer α-miR2392

The present inventors next tested the effectiveness of α-miR2392 in Vero cells infected with SARS-Cov2 at a Multiplicity of infection (MOI) of 0.01. As shown in FIG. 3, Vero cells were infected on day 0, and treatment was started 2 hours before infection for total period of 72 hours. Dose ranges are shown for FASTmer targeting miR2392 (α-miR2392). The blue curve represents percent inhibition normalized according to cell only and the activity of the vehicle controls:

$$( \% inhibiiton = \frac{( drug ) - ( blank )}{( cell only ) - ( blank )} \times 100 )$$

The red curve represents percentage cytotoxicity normalized according to cell-only uninfected controls and media-only controls:

$$( \% TOX = ( 1 - \frac{( drug ) - ( blank )}{( cell only ) - ( blank )} ) \times 100 )$$

As shown in FIG. 3, the FASTmer targeting miR2392 (α-miR2392) demonstrated an inhibitory effect in the cells infected with SARS-Cov2.

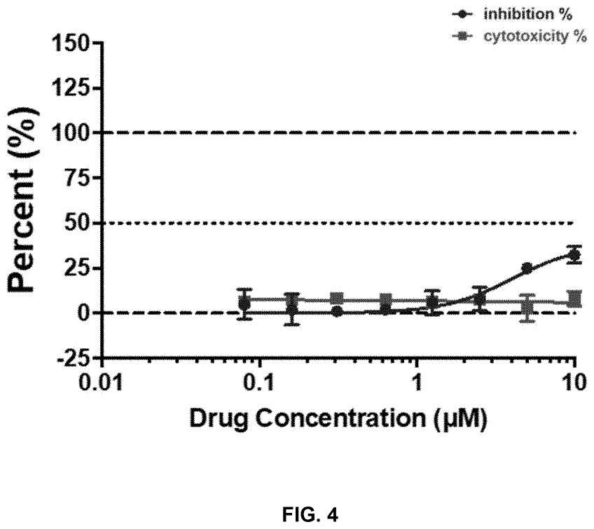

The present inventors next tested the cytotoxicity of α-miR2392 in Vero cells infected with SARS-Cov2 at a Multiplicity of infection (MOI) of 0.01. As shown in FIG. 4, Vero cells were infected on day 0, and treatment was started 2 hours before infection for total period of 72 hours. Dose ranges are shown for nonsense FASTmer control (α-NS). Increasing doses of the FASTmer targeting miR2392 (α-miR2392) did not demonstrate a cytotoxic effect in cells infected with SARS-Cov2.

### Example 6: Evaluation of Toxicity and Efficacy of α-miR2392 in an In Vivo Infection Model

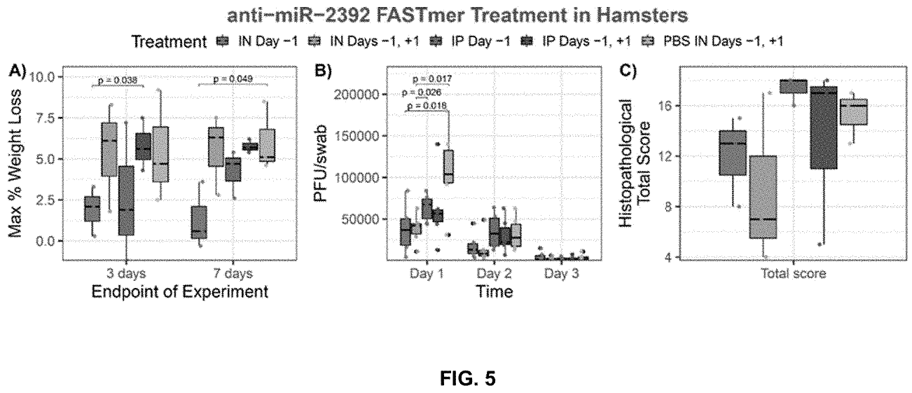

The present inventors tested the efficacy and cytotoxicity of α-miR2392 in an in vivo models, specifically a hamsters infected with SARS-Cov2. As shown in FIG. 5A, the percentage weight loss on day 3 and 7 compared to day 0. The FASTmer treatments have comparable or lower weight loss than PBS control. FIG. 5B, shows plaque-forming units per swab ((PFU/swab) for various FASTmer treatments on day 1, 2 and 3. The FAST-SARSCov2mi IN-1 treatment showed significantly lower than the no treatment (PBS) condition on day 1. FIG. 5C demonstrates that FASTmer treatments have lower histopathological total score than the PBS control.

TABLES
Table 1 List of FASTmer sequences
designed against miR2392

| | | Tar- | | | |
|---|---|---|---|---|---|
| | | get | Hg38 | Hg38 | Notable |
| | Sequence | Loca- | 0 MM | 1 MM | Off- |
| PNA Name | (N to C) | tion# | OT | OT | Targets |
| α-miR2392 | ACCTCTCA | 5-19 | 0 | 23 | |
| v1 | CCCCCAT | | | | |
| (SEQ ID | | | | | |
| NO. 1) | | | | | |
| α-miR2392 | TCTCACCC | 2-16 | 2 | 53 | Lemur |
| v2 | CCATCCT | | | | tyrosine |
| (SEQ ID | | | | | kinase; |
| NO. 2) | | | | | SPT6 |
| | | | | | homolog |
| | | | | | histone |
| | | | | | chaperone |

#On mature human micro-RNA 2392, miRBase: MIMAT0019043
* MM OT: mismatch off-target (e.g. 0 MM OT is a zero-mismatch alignment to a region expected to inhibit protein expression) TCTCACCCCCATCC

### Definitions

For the sake of clarity and readability, the following scientific background information and definitions are provided. Any technical features disclosed thereby can be part of each and every embodiment of the invention. Additional definitions and explanations can be provided in the context of this disclosure.

As used herein, “antisense oligomers” or “antisense oligonucleotide” means any antisense molecule that may modulate the expression of one or more genes. Examples may include antisense PNAs, antisense RNA. This terms also encompasses RNA or DNA oligomers such as interfering RNA molecules, such as dsRNA, dsDNA, mRNA, siRNA, or hpRNA as well as locked nucleic acids, BNA, polypeptides and other oligomers and the like.

As used herein, a “FASTmer,” may include an “oligomer” or “therapeutic oligomer” generated using the FAST Platform as generally described herein. In certain embodiments, a FASTmer may include or “antisense oligonucleotides,” which may include any antisense molecule that may inhibit the activity of one or more host miRNAs. Examples may include anti sense PNAs, or antisense RNA. This term also encompasses RNA or DNA oligomers such as interfering RNA molecules, such as dsRNA, dsDNA, mRNA, siRNA, or hpRNA as well as locked nucleic acids, BNA, polypeptides and other oligomers and the like. In yet another embodiment, the PNA comprises at least one modified phosphate backbone selected from the group consisting of a phosphorothioate, a phosphorodithioate, a phosphoramidothioate, a phosphoramidate, a phosphordiamidate, a methylphosphonate, an alkyl phosphotriester, and a formacetal or analogs thereof.

The term “target sequence,” may mean a nucleotide sequence, such as a miRNA sequence, that may be complementary to antisense molecules, and preferably an antisense FASTmer, such as an antisense PNA FASTmer. It will be recognized by those of skill in the art that any of the sequences herein above can be targeted by antisense inhibitors. Given the benefit of this disclosure, those of skill in the art will be able to identify a target sequence and design an antisense inhibitor oligomer, or FASTmer to target the gene or miRNA sequence. Inhibition can be caused by steric interference resulting from an antisense oligomer binding the DNA or miRNA sequence, thereby preventing proper transcription of the DNA sequence or activity of the miRNA sequence.

The term “homologous” or “sequence identity” as used herein means a nucleic acid (or fragment thereof), including morpholino nucleic acids, or a protein (or a fragment thereof) having a degree of homology to the corresponding natural reference nucleic acid or protein that may be in excess of 70%, or in excess of 80%, or in excess of 85%, or in excess of 90%, or in excess of 91%, or in excess of 92%, or in excess of 93%, or in excess of 94%, or in excess of 95%, or in excess of 96%, or in excess of 97%, or in excess of 98%, or in excess of 99%). For example, in regard to peptides or polypeptides, the percentage of homology or identity as described herein is typically calculated as the percentage of amino acid residues found in the smaller of the two sequences which align with identical amino acid residues in the sequence being compared, when four gaps in a length of 100 amino acids may be introduced to assist in that alignment (as set forth by Dayhoff, in Atlas of Protein Sequence and Structure, Vol. 5, p. 124, National Biochemical Research Foundation, Washington, D.C. (1972)). In one embodiment, the percentage of homology as described above is calculated as the percentage of the components found in the smaller of the two sequences that may also be found in the larger of the two sequences (with the introduction of gaps), with a component being defined as a sequence of four contiguous amino acids. Also included as substantially homologous is any protein product which may be isolated by virtue of cross reactivity with antibodies to the native protein product. Sequence identity or homology can be determined by comparing the sequences when aligned so as to maximize overlap and identity while minimizing sequence gaps. In particular, sequence identity may be determined using any of a number of mathematical algorithms. A non-limiting example of a mathematical algorithm used for comparison of two sequences is the algorithm of Karlin & Altschul, Proc. Natl. Acad. Sci. USA 1990, 87, 2264-2268, modified as in Karlin & Altschul, Proc. Natl. Acad. Sci. USA 1993, 90, 5873-5877.

In another embodiment, the invention includes antisense PNAs that have substantial sequence similarity to the PNAs. Two PNAs have “substantial sequence identity” when both of the PNAs bind to a target sequence.

As used herein, the terms “inhibit” and “inhibition” means to reduce a molecule, a reaction, an interaction, a gene, an mRNA, and/or a protein's expression, stability, function, or activity by a measurable amount, or to prevent such entirely. In one preferred embodiment, the term “inhibit” and “inhibition” means to reduce the stability, function, or activity of a miRNA. “Inhibitors” are compounds that, e.g., bind to, partially or totally block stimulation, decrease, prevent, delay activation, inactivate, desensitize, or down regulate a protein, a gene, and an mRNA stability, expression, function, and activity, e.g., antagonists. In one preferred embodiment, the term “Inhibitors” means an antisense FASTmer that reduces the stability, function, or activity of a miRNA.

As used herein, “host” or “subject” refers to a human or animal subject. In certain preferred embodiments, the subject is a human at risk of infection by COVID-19 or infected with COVID-19. The term “treating”, as used herein, unless otherwise indicated, means reversing, alleviating, inhibiting the progress of, or preventing the disorder or condition to which such term applies, or one or more symptoms of such disorder or condition. The term “treatment”, as used herein, unless otherwise indicated, refers to the act of treating as “treating” is defined immediately above.

As used herein, the terms “complementary” or “complement” also refer to a nucleic acid comprising a sequence of consecutive nucleobases or semi-consecutive nucleobases (e.g., one or more nucleobase moieties are not present in the molecule) capable of hybridizing to another nucleic acid strand or duplex even if less than all the nucleobases do not base pair with a counterpart nucleobase. In certain embodiments, a “complementary” nucleic acid comprises a sequence in which about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%), about 98%), about 99%, or about 100%, and any range derivable therein, of the nucleobase sequence is capable of base-pairing with a single or double stranded nucleic acid molecule during hybridization. In certain embodiments, the term “complementary” refers to a nucleic acid that may hybridize to another nucleic acid strand or duplex in stringent conditions, as would be understood by one of ordinary skill in the art.

The term “composition” or “composition of the invention” generally refers to a FASTmer, and preferably an antisense FASTmer that may be configured to inhibit miRNA-2392 in a host. A “pharmaceutical composition” may include a miRNA inhibitor, or FASTmer of the invention and an agent, e.g., a carrier, that may typically be used within a pharmaceutical composition for facilitating administering of the components of the pharmaceutical composition to an individual.

The term “therapeutically effective amount” as used herein refers to that amount of a FASTmer composition being administered which will relieve to some extent one or more of the symptoms of the disorder being treated. In reference to the treatment of a viral infection, and in particular a COVID-19 infection, a therapeutically effective amount refers to that amount which has the effect of (1) reducing the infection, (2) inhibiting (that is, slowing to some extent, preferably stopping) viral replication, (3) inhibiting to some extent (that is, slowing to some extent, preferably stopping) viral pathogenicity, and/or (4) relieving to some extent (or, preferably, eliminating) one or more signs or symptoms associated with the viral infection. The compositions of the invention can be used for veterinary medical purposes, as a pharmaceutical composition or as a vaccine or treatment. For example, a “therapeutically effective amount” of is a dosage of the compound that is sufficient to achieve a desired therapeutic effect. For example, a therapeutically effective amount of a compound, such as an antisense FASTmer, and preferably an antisense FASTmer targeting configured to inhibit miRNA-2392, may be such that the subject receives a dosage of about 0.1 μg/kg body weight/day to about 1000 mg/kg body weight/day, for example, a dosage of about 1 μg/kg body weight/day to about 1000 μg/kg body weight/day, such as a dosage of about 5 μg/kg body weight/day to about 500 μg/kg body weight/day. In another embodiment, the subject receives a dosage of less than 0.1 μg/kg body weight/day, or more than 1000 mg/kg body weight/day.

In a preferred embodiment, the FASTmer of the (pharmaceutical) composition, and preferably an antisense FASTmer configured to inhibit miRNA-2392 or kit of parts according to the invention is provided in lyophilized form. Preferably, the lyophilized FASTmer is reconstituted in a suitable buffer, advantageously based on an aqueous carrier, prior to administration, e.g., Ringer-Lactate solution, which is preferred, Ringer solution, a phosphate buffer solution. In a preferred embodiment, the (pharmaceutical) composition, the FASTmer or the kit of parts according to the invention contains at least one, two, three, four, five, six or more FASTmer, preferably and preferably antisense FASTmers configured to inhibit miRNA-2392, which are provided separately in lyophilized form (optionally together with at least one further additive) and which are preferably reconstituted separately in a suitable buffer (such as Ringer-Lactate solution) prior to their use so as to allow individual administration of each of the FASTmers.

The composition(s) of the invention may typically contain a pharmaceutically acceptable carrier. The term “pharmaceutically acceptable carrier” as used herein preferably includes the liquid or non-liquid basis of the inventive antisense FASTmer(s). If the inventive antisense FASTmer is provided in liquid form, the carrier will be water, typically pyrogen-free water; isotonic saline or buffered (aqueous) solutions, e.g phosphate, citrate etc. buffered solutions. Particularly for injection of the inventive antisense FASTmer, water or preferably a buffer, more preferably an aqueous buffer, may be used, containing a sodium salt, preferably at least 50 mM of a sodium salt, a calcium salt, preferably at least 0.01 mM of a calcium salt, and optionally a potassium salt, preferably at least 3 mM of a potassium salt. According to a preferred embodiment, the sodium, calcium and, optionally, potassium salts may occur in the form of their halogenides, e.g. chlorides, iodides, or bromides, in the form of their hydroxides, carbonates, hydrogen carbonates, or sulfates, etc. Without being limited thereto, examples of sodium salts include e.g., NaCl, Nal, NaBr, a2C (¼, NaHCCh, a2S0₄, examples of the optional potassium salts include e.g., KCI, KI, KBr, K2CO3, KHCO3, K2SO4, and examples of calcium salts include e.g. CaCb, Ca12, CaBr₂, CaCC>3, CaSC, Ca(OH)₂. Furthermore, organic anions of the aforementioned cations may be contained in the buffer. According to a more preferred embodiment, the buffer suitable for injection purposes as defined above, may contain salts selected from sodium chloride (NaCl), calcium chloride (CaCb) and optionally potassium chloride (KCI), wherein further anions may be present additional to the chlorides. CaCb can also be replaced by another salt like KCI. Typically, the salts in the injection buffer are present in a concentration of at least 50 mM sodium chloride (NaCl), at least 3 mM potassium chloride (KCI) and at least 0.01 mM calcium chloride (CaCb). The injection buffer may be hypertonic, isotonic or hypotonic with reference to the specific reference medium, i.e., the buffer may have a higher, identical or lower salt content with reference to the specific reference medium, wherein preferably such concentrations of the afore mentioned salts may be used, which do not lead to damage of cells due to osmosis or other concentration effects. Reference media are e.g. in “in vivo” methods occurring liquids such as blood, lymph, cytosolic liquids, or other body liquids, or e.g. liquids, which may be used as reference media in “in vitro” methods, such as common buffers or liquids. Such common buffers or liquids are known to a skilled person. Ringer-Lactate solution is particularly preferred as a liquid basis.

However, one or more compatible solid or liquid fillers or diluents or encapsulating compounds may be used as well, which are suitable for administration to a person. Pharmaceutically acceptable carriers, fillers and diluents must, of course, have sufficiently high purity and sufficiently low toxicity to make them suitable for administration to a person to be treated. Some examples of compounds which can be used as pharmaceutically acceptable carriers, fillers or constituents thereof are sugars, such as, for example, lactose, glucose, trehalose and sucrose; starches, such as, for example, corn starch or potato starch; dextrose; cellulose and its derivatives, such as, for example, sodium carboxymethylcellulose, ethylcellulose, cellulose acetate; powdered tragacanth; malt; gelatin; tallow; solid glidants, such as, for example, stearic acid, magnesium stearate; calcium sulfate; vegetable oils, such as, for example, groundnut oil, cottonseed oil, sesame oil, olive oil, corn oil and oil from theobroma; polyols, such as, for example, polypropylene glycol, glycerol, sorbitol, mannitol and polyethylene glycol; alginic acid.

The choice of a pharmaceutically acceptable carrier is determined, in principle, by the manner, in which the pharmaceutical composition or antisense FASTmer according to the invention is administered. The composition or antisense FASTmer can be administered, for example, systemically or locally. Routes for systemic administration in general include, for example, transdermal, oral, parenteral routes, including subcutaneous, intravenous, intramuscular, intraarterial, intradermal and intraperitoneal injections and/or intranasal administration routes. Routes for local administration in general include, for example, topical administration routes but also intradermal, transdermal, subcutaneous, or intramuscular injections or intralesional, intracranial, intrapulmonal, intracardial, and sublingual injections. More preferably, composition or antisense FASTmers according to the present invention may be administered by an intradermal, subcutaneous, or intramuscular route, preferably by injection, which may be needle-free and/or needle injection. Compositions/antisense FASTmers are therefore preferably formulated in liquid or solid form. The suitable amount of the antisense FASTmers or composition according to the invention to be administered can be determined by routine experiments, e.g. by using animal models. Such models include, without implying any limitation, rabbit, sheep, mouse, rat, pig, dog and non-human primate models. Preferred unit dose forms for injection include sterile solutions of water, physiological saline or mixtures thereof. The pH of such solutions should be adjusted to about 7.4. Suitable carriers for injection include hydrogels, devices for controlled or delayed release, polylactic acid and collagen matrices. Suitable pharmaceutically acceptable carriers for topical application include those which are suitable for use in lotions, creams, gels and the like. If the inventive composition or antisense FASTmer is to be administered perorally, tablets, capsules and the like are the preferred unit dose form. The pharmaceutically acceptable carriers for the preparation of unit dose forms which can be used for oral administration are well known in the prior art. The choice thereof will depend on secondary considerations such as taste, costs and storability, which are not critical for the purposes of the present invention, and can be made without difficulty by a person skilled in the art.

The term “including” is used herein to mean, and is used interchangeably with, the phrase “including but not limited to.” The term “or” is used herein to mean, and is used interchangeably with, the term “and/or,” unless context clearly indicates otherwise. The terminology used herein is for describing embodiments and is not intended to be limiting. As used herein, the singular forms “a,” “and” and “the” include plural referents, unless the content and context clearly dictate otherwise. Thus, for example, a reference to “a miRNA” may include a combination of two or more such target miRNAs. Unless defined otherwise, all scientific and technical terms are to be understood as having the same meaning as commonly used in the art to which they pertain.

### REFERENCES

1. WHO Coronavirus Disease (COVID-19) Dashboard (Nov. 9, 2020).
2. Antiviral Drugs That Are Approved or Under Evaluation for the Treatment of COVID-19 (Nov. 9, 2020).
3. K. E. Jones, et al., Global trends in emerging infectious diseases. Nature 451, 990-993 (2008).
4. E. S. Pronker, T. C. Weenen, H. Commandeur, E. H. J. H. M. Claassen, A. D. M. E. Osterhaus, Risk in Vaccine Research and Development Quantified. PLoS One 8, e57755 (2013).
5. J. P. Martinez, F. Sasse, M. Brönstrup, J. Diez, A. Meyerhans, Antiviral drug discovery: broad-spectrum drugs from nature. Nat. Prod. Rep. 32, 29-48 (2015).
6. K. L. Warfield, et al., Gene-specific countermeasures against Ebola virus based on antisense phosphorodiamidate morpholino oligomers. PLoS Pathog. 2, el (2006).
7. Y. Wu, et al., Inhibition of highly pathogenic avian H5N1 influenza virus replication by RNA oligonucleotides targeting NS1 gene. Biochem. Biophys. Res. Commun. 365, 369-374 (2008).
8. B. Li, et al., Using siRNA in prophylactic and therapeutic regimens against SARS coronavirus in Rhesus macaque. Nat. Med. 11, 944-951 (2005).
9. Z. Zeng, et al., A Tat-conjugated Peptide Nucleic Acid Tat-PNA-DR Inhibits Hepatitis B Virus Replication In Vitro and In Vivo by Targeting LTR Direct Repeats of HBV RNA. Mol. Ther.-Nucleic Acids 5, e295 (2016).
10. R. Burrer, et al., Antiviral Effects of Antisense Morpholino Oligomers in Murine Coronavirus Infection Models. J. Virol. 81, 5637 LP-5648 (2007).
11. D.-G. Ahn, et al., Interference of ribosomal frameshifting by antisense peptide nucleic acids suppresses SARS coronavirus replication. Antiviral Res. 91, 1-10 (2011).
12. B. W. Neuman, et al., Inhibition, escape, and attenuated growth of severe acute respiratory syndrome coronavirus treated with antisense morpholino oligomers. J. Virol. 79, 9665-9676 (2005).
13. B. D. Gildea, J. M. Coull, Methods for Modulating the Solubility of Synthetic Polymers (2004).
14. E. L. Hatcher, et al., Virus Variation Resource—improved response to emergent viral outbreaks. Nucleic Acids Res. 45, D482-D490 (2017).
15. K. Cleal, L. He, P. D. Watson, a T. Jones, Endocytosis, intracellular traffic and fate of cell penetrating peptide based conjugates and nanoparticles. Curr Pharm Des 19, 2878-2894 (2013).
16. R. L. Juliano, X. Ming, K. Carver, B. Laing, Cellular Uptake and Intracellular Trafficking of Oligonucleotides: Implications for Oligonucleotide Pharmacology. 24 (2014).

SEQUENCE LISTING

SEQ ID NO. 1
Peptide Nucleic Acid
α-miR2392 v1
Artificial
ACCTCTCACCCCCAT
SEQ ID NO. 2
Peptide Nucleic Acid
α-miR2392 v2
Artificial
TCTCACCCCCATCCT
SEQ ID NO. 3
RNA
human miR2392 target sequence
Homo Sapiens
AUGGGGGUGAGAGGU
SEQ ID NO. 4
RNA
human miR2392
Homo Sapiens
AUGGUCCCUCCCAAUCCAGCCAUUCCUCAGACCAGGUGGCUCCCGAGCCA
CCCCAGGCUGUAGGAUGGGGGUGAGAGGUGCUAG

1-10. (canceled)

11. A pharmaceutical composition comprising at least one antisense FASTmer complementary to a target sequence of a microRNA (miRNA) expressed by said subject, and wherein said antisense oligomer bind to, and inhibits said miRNA, and a pharmaceutically acceptable carrier.

12. The composition of claim 11, wherein said miRNA expressed by said subject comprises a miRNA selected from the group consisting of: miRINA-2392, miRNA-181a-5p, miRNA-10, miRNA-10a-5p, miRNA-2393, miRNA-1-5p, miRNA-34a-5, miRNA-30c-5p, miRNA-29b-3p, miRNA-155-5p, and miRNA-124-3p.

13. The composition of claim 11, wherein said miRNA expressed by said subject comprises the miRNA according to the nucleotide sequence SEQ ID NO. 4.

14. The composition claim 11, wherein said target sequence comprises a target sequence according to the nucleotide sequence SEQ ID NO. 3.

15. The composition of claim 11, wherein said antisense FASTmer comprises an antisense peptide nucleic acid (PNA) oligomer.

16. The composition of claim 15 wherein said antisense PNA oligomer comprises an antisense PNA oligomer having a sequence according to SEQ ID NOs. 1, or 2, or a combination of the same.

17. The composition of claim 11, wherein said antisense FASTmer comprises an antisense FASTmer oligomer having a nucleotide sequence according to SEQ ID NOs. 1, or 2, or a combination of the same.

18. A method of treating or preventing COVID-19 coronavirus infection in a subject in need thereof, comprising the step of administering a therapeutically effective amount of the composition of claim 11.

19-26. (canceled)

27. A pharmaceutical composition comprising at least one antisense peptide nucleic acid (PNA) FASTmer directed to a target sequence of microRNA-2392 (miRINA-2392), and a pharmaceutically acceptable carrier.

28. The composition of claim 27, wherein said microRNA-2392 comprises a miRNA according to the nucleotide sequence SEQ ID NO. 4

29. The composition of claim 27, wherein said target sequence comprises a target sequence according to the nucleotide sequence SEQ ID NO. 3.

30. The composition of claim 27, wherein said antisense PNA oligomer comprises an antisense PNA oligomer having a sequence according to SEQ ID NOs. 1, or 2, or a combination of the same.

31. A method of treating or preventing COVID-19 coronavirus infection in a subject in need thereof, comprising the step of administering a therapeutically effective amount of the composition of claim 27.

32. A composition comprising an antisense peptide nucleic acid (PNA) FASTmer directed to a target sequence of microRNA-2392 (miRINA-2392), wherein said antisense PNA FASTmer comprises a PNA having sequence according to SEQ ID NOs. 1, or 2, or a combination of the same.

33. A method of treating or preventing COVID-19 coronavirus infection in a subject in need thereof, comprising the step of administering a therapeutically effective amount of the composition of claim 32.


SEQUENCE LISTING (4 sequences)

SEQ ID NO: 1 - DNA, 15 bp, Artificial
  miR2392 v1
acctctcacccccat

SEQ ID NO: 2 - DNA, 15 bp, Artificial
  miR2392 v2
tctcacccccatcct

SEQ ID NO: 3 - RNA, 15 nt, Homo Sapiens
augggggugagaggu

SEQ ID NO: 4 - RNA, 84 nt, Homo Sapiens
auggucccucccaauccagccauuccucagaccagguggcucccgagccaccccaggcuguaggaugggggugagaggugcuag